(19) United States
(12) Patent Application Publication
(10) Pub. No.: US 2026/0345691 A1
(43) Pub. Date: Nov. 5, 2026

(54) COMPOSITION AND METHOD FOR PROMOTING HEALTHY FUNCTION OF STEM CELLS

(71) Applicant: Qualia Life Sciences LLC., CARLSBAD, CA (US)

(72) Inventor: Gregory Kelly, Oceanside, CA (US)

(21) Appl. No.: 19/641,903

(22) Filed: Apr. 8, 2026

Related U.S. Application Data
(60) Provisional application No. 63/788,667, filed on Apr. 14, 2025.

(51) Int. Cl. A61K 35/644; A61K 31/192; A61K 31/198; A61K 31/375; A61K 31/555; A61K 31/7072; A61K 31/708; A61K 31/716; A61K 35/748; A61K 36/03; A61K 36/185; A61K 36/232; A61K 36/258; A61K 36/28; A61K 36/481; A61K 36/537; A61K 36/65; A61K 36/886; A61K 38/48; A61P 43/00

(52) U.S. Cl. CPC A61K 35/644; A61K 31/192; A61K 31/198; A61K 31/375; A61K 31/555; A61K 31/7072; A61K 31/708; A61K 31/716; A61K 35/748; A61K 36/03; A61K 36/185; A61K 36/232; A61K 36/258; A61K 36/28; A61K 36/481; A61K 36/537; A61K 36/65; A61K 36/886; A61K 38/4873; A61P 43/00; C12Y 304/22032

(57) ABSTRACT

A composition for supporting stem cell health in humans is provided. A dose of the composition comprises a synergistically effective amount of at least one of sea buckthorn fruit extract, blue-green algae extract, and Aloe macroclada leaf extract. The composition may further comprise a synergistically effective amount of soluble beta glucan, Panax notoginseng root extract, royal jelly powder, L-serine, Okinawa mozuku extract, and nucleotides. Other compositions for supporting stem cell health in humans are provided. Related methods of use and methods of manufacture are also provided.

### CROSS-REFERENCE TO RELATED APPLICATIONS

This application claims the benefit of U.S. Provisional Patent Application No. 63/788,667, filed Apr. 14, 2025, which is hereby incorporated by reference in its entirety.

### FIELD OF THE INVENTION

This disclosure is related to compositions that promote healthy function of stem cells and related methods.

### BACKGROUND OF THE INVENTION

Stem cells, a part of the body's natural repair system, are “blank slate” cells that have the ability to develop into many different types of specialized cells including, but not limited to, bone, joint, muscle and/or blood cells. Stem cells create new cells when needed and replenish and repair damaged tissues throughout the body to maintain and regenerate tissues throughout life.

There are multiple “hallmarks” of aging. Specifically, according to one view, there are 12 such hallmarks of aging: genomic instability, telomere attrition, epigenetic alterations, loss of proteostasis, disabled macroautophagy, deregulated nutrient-sensing, mitochondrial dysfunction, cellular senescence, stem cell exhaustion, altered intercellular communication, chronic inflammation, and dysbiosis.

With stem cell exhaustion, there is a decline in the number and function of stem cells within the body as we age, which leads to a reduced ability to regenerate tissues and repair damage. This, essentially, affects our capacity to heal and rejuvenate tissues, with notable declines occurring by age 40. For example, a recent study of ~17,000 samples over 30 bodily tissues found that in ~60% of those tissues, age negatively correlated with “stemness”, or a stem cell's ability to self-renew and differentiate. Put another way, stem cell health deteriorates across multiple organ systems with age. See, e.g., dos Santos G A, Magdaleno G D V, de Magalhães JP. Evidence of a pan-tissue decline in stemness during human aging. Aging (Albany NY). 2024 Apr. 4; 16:5796-5810. https://doi.org/10.18632/aging.205717.

Accordingly, there is a need for compositions that promote healthy function of stem cells and related methods.

### SUMMARY OF THE INVENTION

In some embodiments, a composition for supporting stem cell health in humans is provided. A dose of the composition comprises a synergistically effective amount of at least one of sea buckthorn fruit extract, blue-green algae extract, and Aloe macroclada leaf extract. In some such embodiments, the composition further comprises a synergistically effective amount of soluble beta glucan, Panax notoginseng (e.g., Tienchi Ginseng) root extract, royal jelly powder, L-serine, Okinawa mozuku extract, and nucleotides.

In some embodiments, the dose of the composition comprises a synergistically effective amount of sea buckthorn fruit extract, blue-green algae extract, Aloe macroclada leaf extract, L-serine, soluble beta glucan, Okinawa mozuku extract, nucleotides, royal jelly powder, ferulic acid, Panax notoginseng root extract, white peony root extract, spirulina extract, Salvia miltiorrhiza root extract, astragalus root extract, bromelain, and/or a cholorophyllin compound. As described anywherein herein, Panax notoginseng may also be known as Tienchi Ginseng. As described anywherein herein, white peony may also be known as Chinese peony. As described anywherein herein, Salvia miltiorrhiza may also be known as Chinese sage.

In some embodiments, the dose of the composition comprises a synergistically effective amount of sea buckthorn fruit extract, blue-green algae extract, Aloe macroclada leaf extract, L-serine, soluble beta glucan, Okinawa mozuku extract, nucleotides, royal jelly powder, ferulic acid, Panax notoginseng root extract, white peony root extract, Salvia miltiorrhiza root extract, astragalus root extract, and/or a cholorophyllin compound.

In some embodiments, the sea buckthorn fruit extract comprises Hippophaeo rhamnoides extract. In some embodiments, the blue-green algae extract comprises Aphanizomenon flos-aquae extract. In some embodiments, the soluble beta glucan comprises baker's yeast soluble beta-1,3/1,6-glucan. In some embodiments, the Okinawa mozuku extract comprises Cladosiphon okamuranus extract. In some embodiments, the nucleotides comprise: uridine monophosphate, or cytidine monophosphate, uridine monophosphate, guanosine monophosphate, and adenosine monophosphate. In some embodiments, the white peony root extract comprises Paeonia lactiflora root extract. In some embodiments, the Salvia miltiorrhiza is a concentrated root extract. In some embodiments, the astragalus root extract comprises Astragalus membranaceus extract. And, in some embodiments, the cholorophyllin compound comprises sodium copper chlorophyllin.

In some embodiments, the synergistically effective amount comprises approximately 50-500 mg of bromelain (270 mg).

In some embodiments, the synergistically effective amount is approximately 250-700 mg of the sea buckthorn fruit extract (500 mg), approximately 300-1500 mg of the blue-green algae extract (400 mg), approximately 200-400 mg of the Aloe macroclada leaf extract (300 mg), approximately 300-3000 mg of the L-serine (300 mg), approximately 50-300 mg of the soluble beta glucan (250 mg), approximately 100-1500 mg of the Okinawa mozuku extract (200 mg), approximately 100-500 mg of the nucleotides (200 mg), approximately 100-500 mg of the royal jelly powder (200 mg), approximately 100-300 mg of the ferulic acid (200 mg), approximately 50-400 mg of the Panax notoginseng root extract (200 mg), approximately 100-500 mg of the white peony root extract (180 mg), less than approximately 50 mg of spirulina extract (40 mg), approximately 100-500 mg of the Salvia miltiorrhiza root extract (150 mg), approximately 50-1000 mg of the astragalus root extract (100 mg), approximately 50-500 mg of the bromelain (100 mg), and approximately 25-300 mg of the sodium copper cholorophyllin (e.g., 2 mg of copper from 50 mg of sodium copper cholorophyllin).

In some embodiments, the synergistically effective amount is approximately 250-700 mg of the sea buckthorn fruit extract (500 mg), approximately 300-1500 mg of the blue-green algae extract (400 mg), approximately 200-400 mg of the Aloe macroclada leaf extract (300 mg), approximately 300-3000 mg of the L-serine (300 mg), approximately 50-300 mg of the soluble beta glucan (250 mg), approximately 100-1500 mg of the Okinawa mozuku extract (200 mg), approximately 100-500 mg of the nucleotides (200 mg), approximately 100-500 mg of the royal jelly powder (200 mg), approximately 100-300 mg of the ferulic acid (200 mg), approximately 50-400 mg of the Panax notoginseng root extract (200 mg), approximately 100-500 mg of the white peony root extract (180 mg), approximately 100-500 mg of the Salvia miltiorrhiza root extract (150 mg), approximately 50-1000 mg of the astragalus root extract (100 mg), approximately 50-500 mg of the bromelain (100 mg), and approximately 25-300 mg of the sodium copper cholorophyllin (e.g., 2 mg of copper from 50 mg of sodium copper cholorophyllin).

In some embodiments, the synergistically effective amount is approximately 250-700 mg of the sea buckthorn fruit extract (500 mg), approximately 300-1500 mg of the blue-green algae extract (400 mg), approximately 200-400 mg of the Aloe macroclada leaf extract (335 mg), approximately 300-3000 mg of the L-serine (300 mg), approximately 50-300 mg of the soluble beta glucan (250 mg), approximately 100-1500 mg of the Okinawa mozuku extract (150 mg), approximately 100-500 mg of the nucleotides (250 mg), approximately 100-500 mg of the royal jelly powder (225 mg), approximately 100-300 mg of the ferulic acid (200 mg), approximately 50-400 mg of the Panax notoginseng root extract (200 mg), approximately 100-500 mg of the white peony root extract (200 mg), approximately 100-500 mg of the Salvia miltiorrhiza root extract (170 mg), approximately 50-1000 mg of the astragalus root extract (180 mg), and approximately 25-300 mg of the sodium copper cholorophyllin (e.g., 2 mg of copper from 50 mg of sodium copper cholorophyllin).

In some embodiments, the synergistically effective amount is approximately 250-700 mg of the sea buckthorn fruit extract (500 mg), approximately 300-1500 mg of the blue-green algae extract (400 mg), approximately 200-400 mg of the Aloe macroclada leaf extract (335 mg), approximately 300-3000 mg of the L-serine (650 mg), approximately 50-300 mg of the soluble beta glucan (250 mg), approximately 100-1500 mg of the Okinawa mozuku extract (200 mg), approximately 100-500 mg of the nucleotides (250 mg), approximately 100-500 mg of the royal jelly powder (225 mg), approximately 100-300 mg of the ferulic acid (200 mg), approximately 50-400 mg of the Panax notoginseng root extract (200 mg), approximately 100-500 mg of the white peony root extract (200 mg), approximately 100-500 mg of the Salvia miltiorrhiza root extract (170 mg), approximately 50-1000 mg of the astragalus root extract (180 mg), approximately 50-500 mg of the bromelain (270 mg), and approximately 25-300 mg of the sodium copper cholorophyllin (e.g., 2 mg of copper from 50 mg of sodium copper cholorophyllin).

In some embodiments, the sea buckthorn fruit extract comprises approximately 150 mg of sea buckthorn proanthocyanidins. In some embodiments, the nucleotides comprise: approximately 24-26% cytidine monophosphate, approximately 24-26% uridine monophosphate, approximately 24-26% guanosine monophosphate, and approximately 24-26% adenosine monophosphate. In some embodiments, the royal jelly comprises 4% 10-hydroxy-2-decenoic acid (10-HDA). In some embodiments, the white peony extract comprises 50% paeoniflorin, the Astragalus membraneacous root extract comprises approximately 5% astragalosides. In some embodiments, the Okinawa mozuku extract comprises at least 85% fucoidan. And, in some embodiments, the Panax notoginseng root extract comprises approximately 5% Notoginsenosides.

In some embodiments, the synergistically effective amounts of the ferulic acid, the astragalus root extract, the Panax notoginseng root extract, the Salvia miltiorrhiza root extract, the white peony root extract and the fucoidan are formulated to support healthy blood circulation.

In some embodiments, the synergistically effective amounts of the sea buckthorn fruit extract, the blue-green algae extract, the Aloe macroclada leaf extract, the Okinawa mozuku extract and the beta glucan are formulated to support improved stem cell mobilization.

In some embodiments, the synergistically effective amounts of the blue-green algae extract, the Okinawa mozuku extract, the beta glucan, and the nucleotides are formulated to support improved stem cell migration.

In some embodiments, the synergistically effective amounts of the L-serine, the chlorophyllin compound, the astragalus root extract, the Panax notoginseng root extract, the white peony root extract, the beta glucan, the Salvia miltiorrhiza root extract, the spirulina extract, the nucleotides, the royal jelly, and the bromelain are formulated to support improved stem cell proliferation.

In some embodiments, the synergistically effective amounts of the L-serine, the chlorophyllin compound, the astragalus root extract, the Panax notoginseng root extract, the white peony root extract, the beta glucan, the Salvia miltiorrhiza root extract, the nucleotides, the royal jelly, and the bromelain are formulated to support improved stem cell proliferation.

In some embodiments, the synergistically effective amounts of the L-serine, the chlorophyllin compound, the ferulic acid, the astragalus root extract, the Panax notoginseng root extract, the white peony root extract, the beta glucan, the spirulina extract, the royal jelly, the bromelain, and the Salvia miltiorrhiza root are formulated to support improved stem cell differentiation.

In some embodiments, the synergistically effective amounts of the L-serine, the ferulic acid, the Panax notoginseng root extract, the nucleotides, the white peony root extract, and Salvia miltiorrhiza root are formulated to support improved stem cell self-renewal.

In some embodiments, the synergistically effective amounts of the L-serine, the astragalus root extract, and the Okinawa mozuku extract are formulated to support improved stem cell survival.

In some embodiments, the composition is further disposed within at least one pharmaceutically acceptable vehicle. In some embodiments, the pharmaceutically acceptable vehicle comprises an oral capsule. In some embodiments, the composition is a powder.

In some embodiments, the composition is formulated to be taken orally: one of the doses per day, for four consecutive days every month or, alternatively, one half of the dose twice per day, for four consecutive days every month.

In some embodiments, the dose of the composition further includes a synergistically effective amount of one or more of: Echinacea purpurea extract, Angelica sinensis root extract, Chlorella growth factor, and Vitamin C. In some such embodiments, the synergistically effective amount is: approximately 100-500 mg of the Echinacea purpurea extract (e.g., 400 mg), approximately 100-500 mg of the Angelica sinensis root extract, approximately 200-750 mg of the Chlorella growth factor (e.g., 200 mg), and approximately 50-500 mg of Vitamin C (e.g., 250 mg as ascorbic acid).

In some embodiments, a method of supporting stem cell health in humans is provided. The method includes orally administering a composition as described anywhere in this disclosure. Such orally administering may include administering (1) one of the doses per day, for four consecutive days every month, or, alternatively, (2) administering one half of the dose twice per day, for four consecutive days every month.

In some embodiments, such a method includes at least one of identifying the individual in need of stem cell health support, evaluating or monitoring the stem cell function of the individual after the composition is orally administered, and observing improved stem cell function compared to before the composition was orally administered to the individual.

In some embodiments, a method of manufacturing a composition for supporting stem cell health in humans is provided. The method includes combining a synergistically effective amount of ingredients as disclosed anywhere in this disclosure. The method may further include disposing the composition within at least one pharmaceutically acceptable vehicle.

### BRIEF DESCRIPTION OF THE FIGURES

FIG. 1 illustrates a summary of effects of using a composition for supporting stem cell functions in vitro, in animals and/or humans, according to some example embodiments;

FIG. 2 illustrates a composition for supporting stem cell functions in animals and/or humans, according to some example embodiments;

FIG. 3 illustrates an ingredients label illustrating amounts of ingredients of a composition for supporting stem cell functions in animals, mammals and/or humans, according to some example embodiments;

FIG. 4 illustrates another ingredients label illustrating amounts of ingredients of another composition for supporting stem cell functions in animals, mammals and/or humans, according to some example embodiments;

FIG. 5 illustrates another ingredients label illustrating amounts of ingredients of another composition for supporting stem cell health on stem cells in animals, mammals and/or humans, according to some example embodiments;

FIG. 6 illustrates a graph showing increases in mobilization of several types of stem cells after dosing sea buckthorn fruit extract;

FIG. 7 illustrates a table showing concentrations of nucleotides in an example nucleotide formulation;

FIG. 8 illustrates a table showing concentrations of Aloin A+B and polysaccharides in an example Aloe macroclada leaf extract formulation;

FIG. 9 illustrates a molecule of an example formulation of a soluble 1,3/1,6-beta glucan;

FIG. 10 illustrates an ingredients list illustrating amounts of ingredients of a composition, according to some example embodiments, for supporting stem cell functions in animals and/or humans, as utilized in a scientific study on the efficacy of the composition;

FIG. 11 illustrates another ingredients list illustrating amounts of ingredients of another composition, according to some example embodiments, for supporting stem cell functions in animals and/or humans, as utilized in the same scientific study also the list of FIG. 10;

FIG. 12 illustrates a flowchart of a method of use for a composition for supporting stem cell health in animals, mammals and/or humans, according to some example embodiments; and

FIG. 13 illustrates a flowchart of a method of manufacture of a composition for supporting stem cell health in animals, mammals and/or humans, according to some example embodiments.

### DETAILED DESCRIPTION

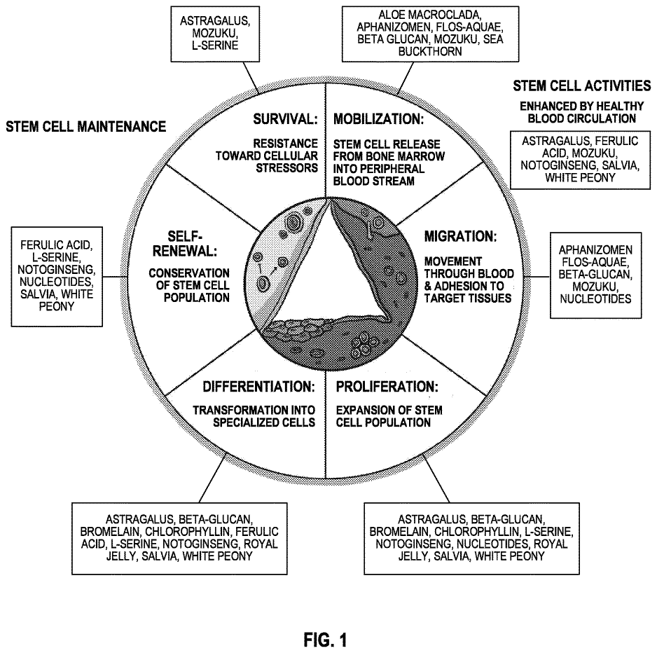

Stem cells primarily migrate to locations within the body that are injured and/or in need of more urgent healing. However, stem cells also migrate anywhere in the body where they can help maintain tissues and/or promote tissue healing. In fact, there are several different types of stem cells, e.g., bone, intestinal, muscle, neural, etc., which participate in repair of wear & tear and/or rejuvenation of body tissues. One goal of the instant disclosure is to provide broad support for mobilization, migration, proliferation, and/or differentiation, collectively referred to as activation of stem cells or stem cells activities, as illustrated in FIG. 1, for a variety of types of stem cells, so as to support and/or improve the body's ability to repair wear & tear and/or rejuvenate tissues throughout the body, as compared to a control condition in which compositions as described anywhere herein are not dosed by or administered to a patient and/or user, or as compared to a control condition in which prior stem cell support formulations, different from those described anywhere herein, are dosed by or administered to the patient and/or user.

Embodiments according to the instant disclosure provide a multi-pronged approach to promoting the healthy activation of stem cells and, thereby, optimize the number available to migrate to, proliferate, and differentiate in tissues that can benefit therefrom. For example, in contrast to prior stem cell support formulations, which are overly focused on single areas of stem cell function (e.g., mobilization, or increasing stem cell numbers), formulations according to any embodiment(s) described herein provide a different, “complex systems” approach, comprising a combination of ingredients specifically formulated to touch and/or affect multiple areas of stem cell health and activity (e.g., stem cell mobilization, migration & homing, proliferation, differentiation, self-renewal, and survival mechanisms, as well as exosome functions).

For example, and not limitation, in some embodiments, traditional Chinese medicine (TCM) herbs that have a reputation for supporting healthy wound healing, blood circulation, and angiogenesis are included in contemplated formulations (e.g., Astragalus membranaceus root extract, Salvia miltiorrhiza root extract) & Panax notoginseng root extract). Some formulations include ferulic acid, which is the main component of Angelica sinensis and provides similar value in TCM as Angelica sinensis. White peony root extract (50% paeoniflorin), fucoidan, and/or echinacea root and herb extract (Echinacea purpurea) have also shown efficacy for supporting healthy angiogenesis.

No other products contain the combination(s) of ingredients of formulations as contemplated anywhere in this disclosure, which may include, but depending on the particular focus of the formulation, are not necessarily limited to, one or more of: Salvia miltiorrhiza, ferulic acid, L-serine, nucleotides, paeoniflorin (50% std. extract from white peony root extract, which has a unique mechanism of CDC42 inhibition worth highlighting), sodium copper chlorophyllin, and/or royal jelly.

By providing a synergistically effective amount of a combination of nutrients that help rejuvenate and fuel the activity of stem cells, and botanicals and compounds to help support those stem cells' mobilization, migration, proliferation, and differentiation, the repair capacity of old tissues may be enhanced to a greater efficiency than has previously been accomplished, thereby, providing an opportunity to boost both health span and lifespan. This unique combination of components provides a greater than additive effect on stem cell promotion.

As will be described in more detail below, compositions 100 comprise a synergistically effective amount of a combination of nutrients that help rejuvenate and fuel the activity of stem cells, and botanicals and other compounds to help support those stem cells' mobilization, migration, proliferation, and differentiation in animals and/or humans. Such compositions are configured to synergistically enrich the body's healing potential, support healthy stem cell activity and function, support youthful tissue repair, optimize muscle and tissue repair, facilitate the body's natural healing and recovery functions, promote healthy recovery from sports-related injuries, and support mental clarity and overall vitality. Moreover, such compositions 100 may accomplish outcomes described anywhere herein at least in part by affecting one or more of: supporting and/or promoting optimal circulating stem cell numbers and activity, supporting and/or promoting healthy stem cell migration and homing, supporting and/or promoting healthy stem cell proliferation, supporting and/or promoting healthy stem cell differentiation, supporting and/or promoting healthy stem cell survival mechanisms, supporting and/or promoting natural stem cell self-renewal, supporting and/or promoting healthy exosome function, supporting and/or promoting healthy blood circulation, supporting and/or promoting immune system resilience, supporting and/or promoting telomere health, supporting and/or promoting mitochondrial health, and/or by helping to protect against oxidative stress.

For example, as shown in FIG. 1, such compositions 100 are specifically formulated to simultaneously support at least 6 aspects of healthy stem cell function—(1) survival, (2) self-renewal, (3) mobilization, (4) migration, (5) proliferation and (6) differentiation—by combining a blend of herbal, algal, and yeast extracts, superfoods and/or stem cell nutrients. FIG. 1 identifies mobilization, migration, proliferation, and differentiation as stem cell activities, and self-renewal and survival as aspects of stem cell maintenance.

In some embodiments, such stem cell activities may be enhanced by promoting and/or supporting healthy blood circulation. For example, a composition 100 may support healthy blood circulation by including a synergistically effective amount of one or more of: ferulic acid, astragalus root extract (e.g., Astragalus membranaceus), Panax notoginseng root extract, Salvia miltiorrhiza root extract, white peony root extract (e.g., Paeonia lactiflora) and/or fucoidan from a brown seaweed such as Mozuku (Cladosiphon okamuranus).

In some embodiments, stem cell mobilization includes stem cell release from bone marrow into the peripheral blood stream. A composition 100 may include a synergistically effective amount of one or more of: sea buckthorn fruit extract (e.g., Hippophae rhamnoides), Aphanizomenon flos-aquae (e.g., blue-green algae extract), Aloe macroclada leaf extract, Okinawa mozuku extract (Cladosiphon okamuranus) and/or soluble 1,3/1,6-beta glucan (e.g., soluble as opposed to insoluble yeast beta glucan) to promote and/or support improved stem cell mobilization.

In some embodiments, stem cell migration includes movement through the blood stream and/or adhesion to target tissues. A composition 100 may include a synergistically effective amount of one or more of: Aphanizomenon flos-aquae (e.g., blue-green algae extract), Okinawa mozuku extract (Cladosiphon okamuranus), soluble 1,3/1,6-beta glucan, and/or nucleotides (e.g., uridine monophosphate or a mix combining 2 or more of the 4 nucleotides) to promote and/or support improved stem cell migration.

In some embodiments, stem cell proliferation includes expansion of the stem cell population. A composition 100 may include a synergistically effective amount of one or more of: L-serine, chlorophyllin (e.g., sodium copper Chlorophyllin), astragalus root extract (e.g., Astragalus membranaceus), Panax notoginseng root extract, white peony root extract (e.g., Paeonia lactiflora), soluble 1,3/1,6-beta glucan (e.g., yeast beta glucan), Salvia miltiorrhiza root extract, nucleotides, bromelain, and/or royal jelly to promote and/or support improved stem cell proliferation. In some embodiments, chlorella growth factor (e.g., Chlorella vulgaris) is also, or alternatively to one or more of the above ingredients, included to promote and/or support improved stem cell proliferation. In yet other embodiments, spirulina is also, or alternatively to one or more of the above ingredients, included to promote and/or support improved stem cell proliferation.

In some embodiments, stem cell differentiation includes transformation of undifferentiated stem cells into specialized cells. A composition 100 may include a synergistically effective amount of one or more of: L-serine, chlorophyllin (e.g., sodium copper Chlorophyllin), ferulic acid, astragalus root extract (e.g., Astragalus membranaceus), Panax notoginseng root extract, white peony root extract (e.g., Paeonia lactiflora), soluble 1,3/1,6-beta glucan (e.g., yeast beta glucan), bromelain, royal jelly, and/or Salvia miltiorrhiza root extract to promote and/or support improved stem cell differentiation. In some embodiments, spirulina is also, or alternatively to one or more of the above ingredients, included to promote and/or support improved stem cell differentiation.

In some embodiments, stem cell self-renewal includes conservation of the stem cell population. A composition 100 may include a synergistically effective amount of one or more of: L-serine, ferulic acid, Panax notoginseng root extract, nucleotides, white peony root extract (e.g., Paeonia lactiflora), and/or Salvia miltiorrhiza root extract to promote and/or support improved stem cell self-renewal. In some embodiments, chlorella growth factor (e.g., Chlorella vulgaris) is also, or alternatively to one or more of the above ingredients, included to promote and/or support improved stem cell self-renewal.

In some embodiments, stem cell survival includes resistance toward cellular stressors. A composition 100 may include a synergistically effective amount of one or more of: astragalus root extract (e.g., Astragalus membranaceus), L-serine, and/or Okinawa mozuku extract (Cladosiphon okamuranus) to support improved stem cell survival. In some embodiments, ferulic acid is also, or alternatively to one or more of the above ingredients, included to promote and/or support improved stem cell survival. In some embodiments, chlorella growth factor (e.g., Chlorella vulgaris) is also, or alternatively to one or more of the above ingredients, included to promote and/or support improved stem cell survival.

### Intermittent Dosing Schedule(s)

Unlike other stem cell support products, which recommend uninterrupted daily supplementation that is expected to cause physiological adaptation and/or stem cell exhaustion over time, the instant disclosure contemplates compositions 100, as described anywhere herein, specifically formulated for an intermittent dosing schedule. For example, and not limitation, depending on the formulation, dosing may be carried out once (or in two divided doses) per day for 2 consecutive days, 3 consecutive days, 4 consecutive days, 5 consecutive days or 6 consecutive days. Depending on the number of consecutive days of dosing, and the size of the daily dose, this multiple consecutive day dosing schedule is specifically formulated to be repeated between twice per month and once every 3 months, for example, being repeated twice per month, once per month, once every 6 weeks, once every 2 months, and/or once every 3 months.

As a preferred example, a composition 100, as described anywhere herein, may be dosed once per day for 4 consecutive days, with this dosing schedule being repeated once a month. Each daily dose could also be split into two divided doses to be taken at temporally spaced times on the same day (e.g., morning and night). The inventor(s) have found such an intermittent approach to make more sense physiologically, and to be more effective compared to continuous daily dosing, if the goal is not the continuous promotion of the mobilization of stem cells, which may, undesirably, result in physiological adaptation and/or exhaustion over time.

### Example Compositions

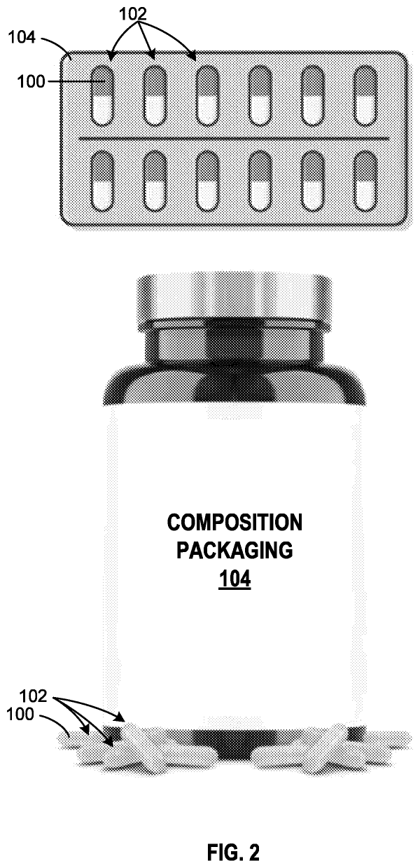

In some embodiments, composition 100 is disposed within at least one pharmaceutically acceptable vehicle 102, for example, capsule form (e.g., oral capsule form) and, in some embodiments, further packaged in packaging 104, which is shown as a bottle in one example at the bottom of FIG. 2, but which may alternatively be any suitable packaging such as blister packs, as shown as another example at the top of FIG. 2, pouches and/or individual servings packaged in sealed bags.

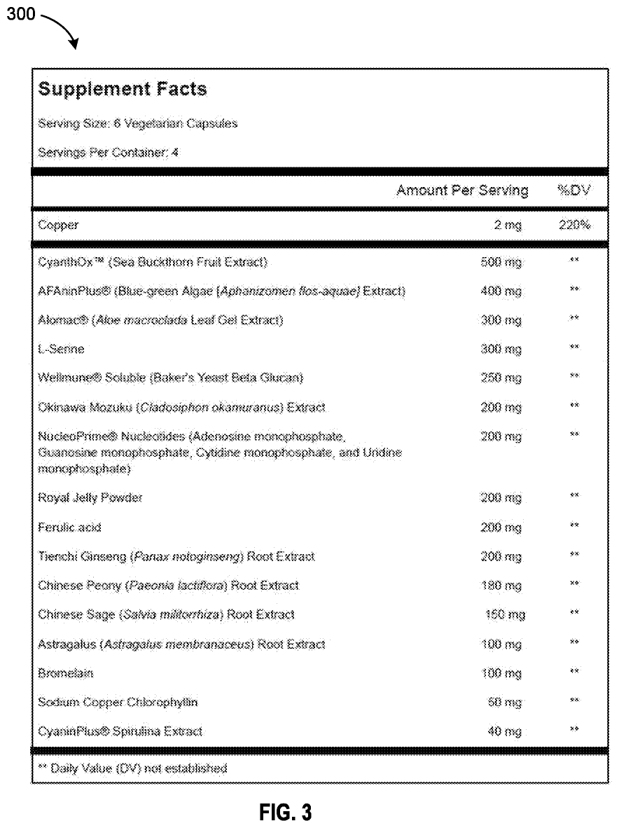

In FIG. 3, an example embodiment of composition 100 is illustrated to have a daily dose of a synergistically effective amount of ingredients as listed on example label 300, divided between 6 capsules 102. This daily dose of 6 capsules 102 may be dosed once per day for 4 consecutive days or, alternatively in 2 divided doses of 3 capsules 102 taken at two different times on the same day, for the same 4 consecutive days. According to label 300, this 4-day dosing schedule is repeated once per month. However, not all embodiments are so limited and, depending on the desired outcome and/or particulars of the patient and/or user, alternative dosing schedules are also contemplated, for example and not limitation, dosing once (or in two divided doses) per day for 2 consecutive days, 3 consecutive days, 4 consecutive days, 5 consecutive days or 6 consecutive days.

According to such embodiments, label 300 illustrates that a dose of composition 100 includes: 500 mg of CyanthOx™ (Sea Buckthorn Fruit Extract), 400 mg of AFAninPlus® (Blue-green Algae [Aphanizomenon flos-aquae] Extract), 300 mg of Alomac® (Aloe macroclada Leaf Gel Extract), 300 mg of L-Serine, 250 mg of Wellmune® Soluble (Baker's Yeast Beta Glucan), 200 mg of Okinawa Mozuku (Cladosiphon okamuranus) Extract, 200 mg of Nucleoprime™ Nucleotides, 200 mg of Royal Jelly Powder, 180 mg of white peony (Paeonia lactiflora) Root Extract, 200 mg of ferulic acid, 150 mg of Salvia miltiorrhiza Root Extract, 200 mg of Panax Notoginseng Root Extract, 100 mg of Astragalus (Astragalus membranaceus) Root Extract, 100 mg of bromelain, 50 mg of Sodium Copper Chlorophyllin, and 40 mg of CyaninPlus® Spirulina Extract. In some embodiments, composition 100 does not include stem cells or compounds derived from stem cells. While label 300 illustrates a particular formulation of composition 100, the present disclosure is not so limited and contemplates substantially similar, or substantially different synergistically effective amounts of one or more of the above-described ingredients compared to that set forth in label 300.

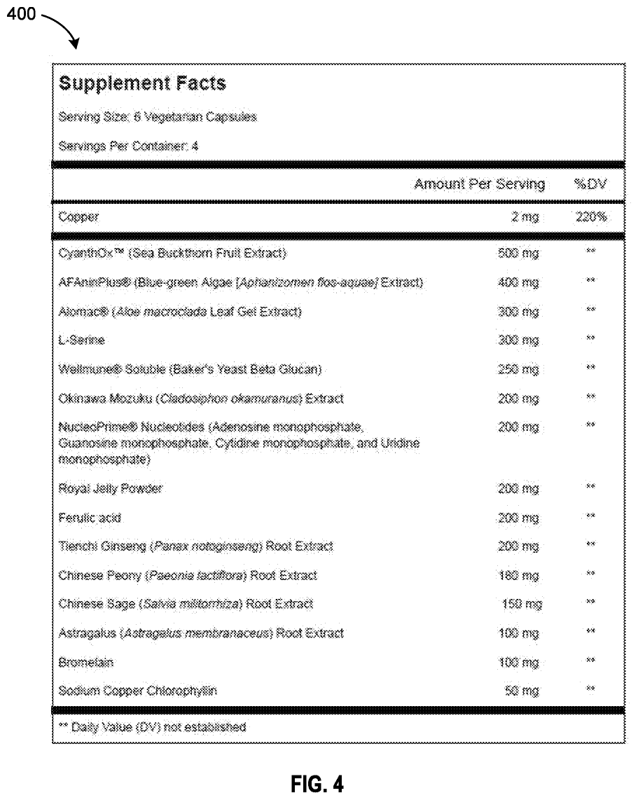

According to other such embodiments, as shown in FIG. 4, a label 400 illustrates that a dose of composition 100 includes: 500 mg of CyanthOx™ (Sea Buckthorn Fruit Extract), 400 mg of AFAninPlus® (Blue-green Algae [Aphanizomenon flos-aquae] Extract), 300 mg of Alomac® (Aloe macroclada Leaf Gel Extract), 300 mg of L-Serine, 250 mg of Wellmune® Soluble (Baker's Yeast Beta Glucan), 200 mg of Okinawa Mozuku (Cladosiphon okamuranus) Extract, 200 mg of Nucleoprime™ Nucleotides, 200 mg of Royal Jelly Powder, 200 mg of ferulic acid, 200 mg of Panax Notoginseng Root Extract, 180 mg of white peony (Paeonia lactiflora) Root Extract, 150 mg of Salvia miltiorrhiza Root Extract, 100 mg of Astragalus (Astragalus membranaceus) Root Extract, 100 mg of bromelain and 50 mg of Sodium Copper Chlorophyllin. In some embodiments, composition 100 does not include stem cells or compounds derived from stem cells. While label 400 illustrates a particular formulation of composition 100, the present disclosure is not so limited and contemplates substantially similar, or substantially different synergistically effective amounts of one or more of the above-described ingredients compared to that set forth in label 400.

For example, and not limitation, TABLE 1, below, illustrates ranges of exemplary (e.g., example) amounts of each of several ingredients, as well as one or more stem cell functions each ingredient is included to improve and/or support, in a daily dose of a composition, such as compositions 100. As described in this disclosure, a composition comprising amounts of two or more (e.g., in some embodiments, each) of the ingredients listed in TABLE 1 within the exemplary amounts ranges may be disposed within at least one pharmaceutically acceptable vehicle 102, for example, capsule form (e.g., oral capsule form).

TABLE 1

| | Exemplary | |
|---|---|---|
| Ingredient | Amounts (mg) | Stem Cell Function |
| Sea Buckthorn Fruit Extract | 250-700 | Stem cell mobilization |
| (such as CyanthOx ™) | | |
| Soluble Beta Glucan (such | 50-300 | Stem cell mobilization, |
| as Wellmune ® Soluble Baker's | | migration, proliferation, |
| Yeast Beta Glucan) | | and differentiation |
| Panax notoginseng Root Extract | 50-400 | Stem cell proliferation, |
| | | differentiation, and self- |
| | | renewal |
| Royal Jelly Powder | 100-500 | Stem cell proliferation and |
| | | differentiation |
| L-Serine | 300-3000 | Stem cell self-renewal, |
| | | survival, and proliferation |
| Brown seaweed extract standardized for | 100-1500 | Stem cell mobilization, |
| fucoidan (such as Okinawa Mozuku | | migration, and survival |
| (Cladosiphon okamuranus) Extract) | | |
| Nucleotides (such as uridine | 100-500 | Stem cell migration, |
| monophosphate alone, or a mix of 4 | | proliferation, and self- |
| nucleotides such as Nucleoprime ™ | | rejuvenation |
| Nucleotides) | | |

As yet another example, and not a limitation, TABLE 2, below, also illustrates ranges of exemplary (e.g., example) amounts of each of several ingredients, as well as one or more stem cell functions each ingredient is included to improve and/or support, in a daily dose of a composition, such as compositions 100. As described in this disclosure, a composition having amounts of two or more (e.g., in some embodiments, each) of the ingredients listed in TABLE 2 (except having at least one of the a-c ingredients listed in the first row) within the exemplary amounts ranges may be disposed within at least one pharmaceutically acceptable vehicle 102, for example, capsule form (e.g., oral capsule form).

TABLE 2

| | Exemplary | |
|---|---|---|
| Ingredient | Amounts (mg) | Stem Cell Function |
| At least one of a, b, or c: | (a) 250-700 | (a) Stem cell |
| (a) Sea Buckthorn Fruit Extract | (b) 300-1500 | mobilization |
| (such as CyanthOx ™) | (c) 200-400 | (b) Stem cell |
| (b) Blue-green Algae [Aphanizomen | | mobilization |
| flos-aquae] Extract (such as | | (c) Stem cell |
| AFAninPlus ®) | | mobilization |
| (c) Aloe macroclada Leaf Gel | | |
| Extract (such as Alomac ®) | | |
| Soluble Beta Glucan (such | 50-300 | Stem cell mobilization, |
| as Wellmune ® Soluble Baker's | | migration, proliferation, |
| Yeast Beta Glucan) | | and differentiation |
| Panax notoginseng Root Extract | 50-400 | Stem cell proliferation, |
| | | differentiation, and self- |
| | | renewal |
| Royal Jelly Powder | 100-500 | Stem cell proliferation and |
| | | differentiation |
| L-Serine | 300-3000 | Stem cell self-renewal, |
| | | survival, and proliferation |
| Brown seaweed extract standardized for | 100-1500 | Stem cell mobilization, |
| fucoidan (such as Okinawa Mozuku | | migration, and survival |
| (Cladosiphon okamuranus) Extract) | | |
| Nucleotides (such as uridine | 100-500 | Stem cell migration, |
| monophosphate alone or a mix of 4 | | proliferation, and self- |
| nucleotides such as Nucleoprime ™ | | rejuvenation |
| Nucleotides) | | |

As yet another example, and not a limitation, TABLE 3, below, also illustrates ranges of exemplary (e.g., example) amounts of each of several ingredients, as well as one or more stem cell functions each ingredient is included to improve and/or support, in a daily dose of a composition, such as compositions 100. As described in this disclosure, a composition having amounts of two or more (e.g., in some embodiments each) of the ingredients listed in TABLE 3 within the exemplary amounts ranges may be disposed within at least one pharmaceutically acceptable vehicle 102, for example, capsule form (e.g., oral capsule form). In some embodiments, formulations of composition 100 according to TABLE 3 may be a preferred embodiment, at least in that the ingredients on label 300 of FIG. 3 (which include the listed spirulina extract) and/or on label 400 of FIG. 4 (which do not include the listed spirulina extract) each fall within the ranges of TABLE 3.

TABLE 3

| | Exemplary | |
|---|---|---|
| Ingredient | Amounts (mg) | Stem Cell Function |
| CyanthOxTM (Sea Buckthorn Fruit Extract) | 250-700 | Stem cell mobilization |
| Blue-green Algae [Aphanizomen flos- | 300-1500 | Stem cell mobilization and |
| aquae] Extract (such as AFAninPlus ®) | | migration |
| Aloe macroclada Leaf Gel Extract | 200-400 | Stem cell mobilization |
| (such as Alomac ®) | | |
| L-Serine | 300-3000 | Stem cell self-renewal, |
| | | survival, and proliferation |
| Soluble Beta Glucan (such | 50-300 | Stem cell mobilization, |
| as Wellmune ® Soluble Baker's | | migration, proliferation, and |
| Yeast Beta Glucan) | | differentiation |
| Brown seaweed extract standardized for | 100-1500 | Stem cell mobilization, |
| fucoidan (such as Okinawa Mozuku | | migration, and survival |
| (Cladosiphon okamuranus) Extract) | | |
| Nucleotides (such as uridine | 100-500 | Stem cell migration, |
| monophosphate alone or a mix of 4 | | proliferation, and self- |
| nucleotides such as Nucleoprime ™ | | rejuvenation |
| Nucleotides) | | |
| Royal Jelly Powder | 100-500 | Stem cell proliferation and |
| | | differentiation |
| Ferulic Acid | 100-300 | Stem cell differentiation and |
| | | self-renewal |
| Panax notoginseng Root Extract | 50-400 | Stem cell proliferation, |
| | | differentiation, and self- |
| | | renewal |
| White Peony (Paeonia lactiflora) Root | 100-500 | Stem cell proliferation, |
| Extract standardized for paeoniflorin | | differentiation, and self- |
| | | renewal |
| Salvia miltiorrhiza Root Extract | 100-500 | Stem cell proliferation, |
| | | differentiation, and self- |
| | | renewal |
| Astragalus membranaceus Root Extract | 50-1000 | Stem cell proliferation, |
| | | differentiation, and survival |
| Bromelain | 50-500 | Stem cell proliferation and |
| | | differentiation |
| Chlorophyllin (such as Sodium Copper or | 25-300 | Stem cell proliferation and |
| Sodium Magnesium Chlorophyllin) | | differentiation |
| Spirulina Extract (such as CyaninPlus ®) | <50 | Stem cell proliferation and |
| | (e.g., 40) | differentiation |

As yet another example, and not a limitation, TABLE 4, below, also illustrates ranges of exemplary (e.g., example) amounts of each of several ingredients, including additional ingredients compared to TABLE 3, above, as well as one or more stem cell functions each ingredient is included to improve and/or support, in a daily dose of a composition, such as compositions 100. As described in this disclosure, a composition having amounts of two or more (e.g., in some embodiments each) of the ingredients listed in TABLE 4 within the exemplary amounts ranges may be disposed within at least one pharmaceutically acceptable vehicle 102, for example, capsule form (e.g., oral capsule form). While several embodiments illustrate one or more combinations of ingredients, within exemplary ranges and/or preferred amounts within those ranges, the present disclosure is not so limited and contemplates compositions including any one or more of any ingredients disclosed in any label, table, claim, figure and/or written description as described anywhere herein.

TABLE 4

| | Exemplary | |
|---|---|---|
| Ingredient | Amounts (mg) | Stem Cell Function |
| CyanthOx ™ (Sea Buckthorn Fruit | 250-700 | Stem cell mobilization |
| Extract) | | |
| L-Serine | 300-3000 | Stem cell self-renewal, |
| | | survival, and proliferation |
| Blue-green Algae [Aphanizomen flos- | 300-1500 | Stem cell mobilization and |
| aquae] Extract (such as AFAninPlus ®) | | migration |
| Echinacea purpurea Extract (herb, root, | 100-500 | Stem cell mobilization and |
| or herb and root) | | migration |
| Aloe macroclada Leaf Gel Extract (such | 200-400 | Stem cell mobilization |
| as Alomac ®) | | |
| Astragalus membranaceus Root Extract | 50-1000 | Stem cell proliferation, |
| | | differentiation, and survival |
| Nucleotides (such as uridine | 100-500 | Stem cell migration, |
| monophosphate alone or a mix of 4 | | proliferation, and self- |
| nucleotides such as Nucleoprime ™ | | rejuvenation |
| Nucleotides) | | |
| Soluble Beta Glucan (such as | 50-300 | Stem cell mobilization, |
| Wellmune ® Soluble Baker's | | migration, proliferation, and |
| Yeast Beta Glucan) | | differentiation |
| Royal Jelly Powder | 100-500 | Stem cell proliferation and |
| | | differentiation |
| Brown seaweed extract standardized for | 100-1500 | Stem cell migration and |
| fucoidan (such as Okinawa Mozuku | | survival |
| (Cladosiphon okamuranus) Extract) | | |
| Chlorella Growth Factor | 200-750 | Stem cell proliferation and |
| | | differentiation |
| White Peony (Paeonia lactiflora) Root | 100-500 | Stem cell proliferation, |
| Extract standardized for paeoniflorin | | differentiation, and self- |
| | | renewal |
| Ferulic Acid | 100-300 | Stem cell differentiation and |
| | | self-renewal |
| Salvia miltiorrhiza Root Extract | 100-500 | Stem cell proliferation, |
| | | differentiation, and self- |
| | | renewal |
| Chlorophyllin (such as Sodium Copper | 25-300 | Stem cell proliferation and |
| or Sodium Magnesium Chlorophyllin, | | differentiation |
| e.g., 6 mg of copper from 150 mg of | | |
| sodium copper chlorophyllin) | | |
| Bromelain | 50-500 | Stem cell proliferation and |
| | | differentiation |
| Panax notoginseng Root Extract | 50-400 | Stem cell proliferation, |
| | | differentiation, and self- |
| | | renewal |
| Bromelain | 50-500 | Stem cell proliferation and |
| | | differentiation |
| Vitamin C (e.g., as ascorbic acid) | 50-500 | Stem cell migration, |
| | | proliferation, differentiation, |
| | | and self-rejuvenation |

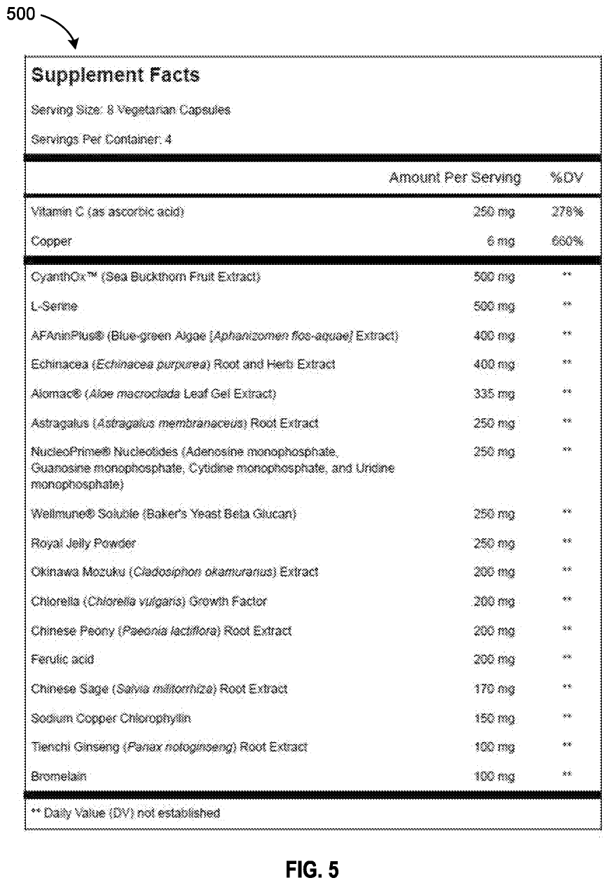

FIG. 5 illustrates a daily dose of yet another example embodiment of composition 100 comprising a synergistically effective amount of ingredients as listed on example label 400, divided between 8 capsules 102. This daily dose of 8 capsules 102 may be dosed once per day for 4 consecutive days or, alternatively in 2 divided doses of 4 capsules 102 each, taken at two different times on the same day, for the same 4 consecutive days. According to label 400, this 4-day dosing schedule is repeated once per month.

According to such embodiments, label 500 illustrates that a dose of composition 100 includes: 500 mg of CyanthOx™ (Sea Buckthorn Fruit Extract), 500 mg of L-serine, 400 mg of AFAninPlus® (Blue-green Algae [Aphanizomen flos-aquae] Extract), 400 mg of Echinacea (Echinacea purpurea) Root and Herb Extract, 335 mg of Alomac® (Aloe macroclada Leaf Extract), 250 mg of Astragalus (Astragalus membranaceus) Root Extract, 250 mg of Nucleoprime™ Nucleotides, 250 mg of Wellmune® Soluble (Baker's Yeast Beta Glucan), 250 mg of Royal Jelly Powder, 200 mg of Okinawa Mozuku (Cladosiphon okamuranus) Extract, 200 mg of Chlorella (Chlorella vulgaris) Growth Factor, 200 mg of White Peony (Paeonia lactiflora) Root Extract, 200 mg of Ferulic acid, 170 mg of Salvia miltiorrhiza Root Extract, 150 mg of Sodium Copper Chlorophyllin (e.g., 6 mg copper), 100 mg of Panax Notoginseng Root Extract, 100 mg of Bromelain, and 250 mg of Vitamin C (e.g., as ascorbic acid). In some embodiments, composition 100 does not include stem cells or compounds derived from stem cells. While label 500 illustrates a particular formulation of composition 100, the present disclosure is not so limited and contemplates substantially similar, or substantially different synergistically effective amounts of one or more of the above-described ingredients compared to that set forth in label 500.

All formulations described and/or contemplated by any portion of this disclosure support and/or enhance the natural, innate functions of stem cells, tissue repair, and/or healing. Longer-term use, e.g., 3 months of supplementation according to a dosing schedule described herein, is expected to rejuvenate these key areas. Composition 100 supports skin health, such as reduced wrinkles and fine lines, improved skin texture, elasticity and a more youthful appearance. Composition 100 supports hair growth, and thicker and healthier hair. Composition 100 supports joint function, joint mobility and comfort. Composition 100 supports muscle and tendon function, tendon and muscle renewal and endurance. Composition 100 supports cognitive function, innate brain cellular rejuvenation processes and regenerative mechanisms. Composition 100 supports overall vitality, energy levels and general feeling of well-being with age. Composition 100 also supports healthy stem cell activity and function, as well as youthful tissue repair. Composition 100 allows users to optimize muscle and tissue repair, facilitate the body's natural healing and recovery, and help aid recovery from sports-related injuries, thereby allowing users to gain more from their workouts.

Specifically, formulations of composition 100 provide multiple ingredients that support stem cell mobilization, multiple ingredients that support stem cell migration and/or homing, multiple ingredients that support stem cell proliferation, multiple ingredients that support stem cell differentiation, multiple ingredients that support improved circulation, multiple ingredients that support stem cell self-renewal, and multiple ingredients that support stem cell survival. Accordingly, in combination, the ingredients of composition(s) 100 are specially formulated to work together to synergistically promote healthy circulating stem cell number, activity, migration & homing, proliferation, differentiation, survival mechanisms, self-renewal, healthy blood circulation, formation of new blood vessels, immune system resilience, exosomal health, telomere health, mitochondrial health, as well as encouraging innate tissue regeneration in individuals identified or known to be in need thereof.

Discussion of several of the individual ingredients in one or more formulations of composition 100 follow below.

### Sea Buchhorn Fruit Extract (Such as CyanthOx™)

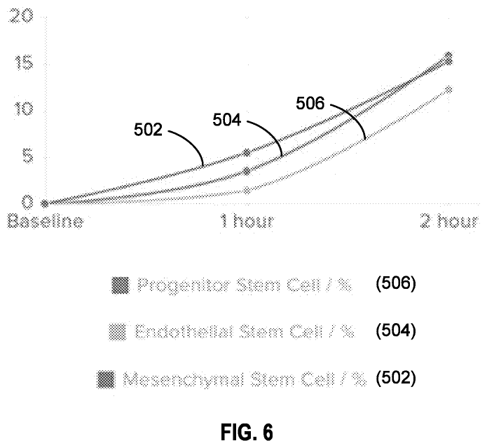

Sea buckthorn fruit are native to Tibet. Sea buckthorn fruit extract is a next generation antioxidant, which mobilizes, and hence increases in blood circulation, at least 3 types of stem cells, contributing to healthy aging, healthy regulation of inflammation, improved skin health, improved vessel health and/or improved blood circulation. Specifically, 150 mg of sea buckthorn proanthocyanidins from 500 mg of CyanthOx™ leads to an increase in mobilization of progenitor stem cells, endothelial stem cells, and lymphocyte mesenchymal stromal cells, by up to 25% 2 hours after consumption of a single dose in humans, thereby supporting preventive health, regenerative health, and postponing the aging process. See, e.g., FIG. 6 and Drapeau C, Benson K F, Jensen G S. Rapid and selective mobilization of specific stem cell types after consumption of a polyphenol-rich extract from sea buckthorn berries (Hippophae) in healthy human subjects. Clin Interv Aging. 2019; 14:253-263. Sea buckthorn proanthocyanidins also enhance collagen production and cross-linkage, support healthy lipid, blood sugar and nitric oxide levels, support healthy retinal photo-oxidation response, and promote gastric ulcer recovery and intestinal integrity.

Blue Green Algae (Aphanizomenon flos-Aquae [AFA] Extract (Such as AFAninPlus®)

AFAninPlus® is an extract derived from Aphanizomenon flos-aquae (AFA), a blue-green algae harvested from the pristine, high-desert Klamath Lake in Southern Oregon. Clinically, AFA has been shown to mobilize and hence boost levels of circulating hematopoietic stem cells by 25%, promote a healthy immune response, promote positive neuroprotective activity, support improved cognitive function, and promote antioxidant protection against free radicals, which are essential for the healthy activities and survival mechanisms of stem cells. See, e.g., Jensen G S, Hart A N, Zaske L A M, et al. Mobilization of human CD34+CD133+ and CD34+CD133− stem cells in vivo by consumption of an extract from Aphanizomenon flos-aquae—related to modulation of CXCR4 expression by an L-selectin ligand? Cardiovascular Revascularization Medicine. 2007; 8 (3): 189-202. AFAninPlus® is halal, kosher, organic, vegan, gluten-free, and non-GMO.

Uridine Monophosphate and/or a Mix of Nucleotides (Such as Nucleoprime™ Nucleotides

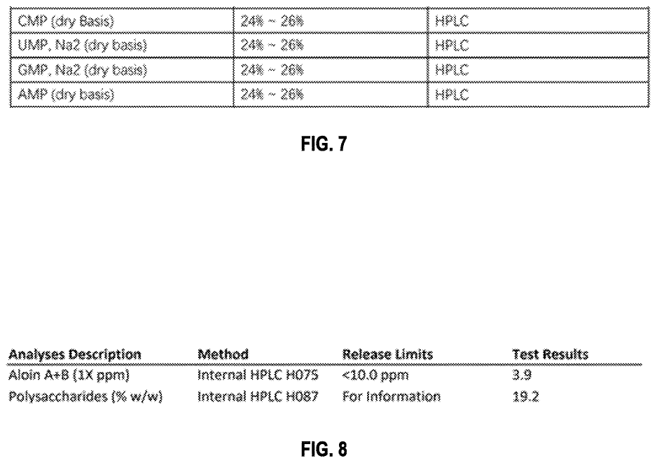

Nucleoprime™ Nucleotides are a clinically researched source of the 4 complex molecules that form the DNA and RNA crucial for healthy stress resilience, act as signaling molecules and metabolic co-factors, and form the body's energy-rich molecules, namely adenosine triphosphate (ATP), which uphold numerous stem cell functions, including their self-renewal mechanisms. Although it can make nucleotides, the body's requirements increase markedly during growth and stress—such as exercise—and in these situations, supplementation with dietary nucleotides may provide tremendous metabolic, immunological and physiological benefits. For example, supplementing nucleotides can prevent declines in peak power performance, support faster recovery and maintenance of force production, promote improved endurance performance, and may improve brain cell health and growth effect. Supplementing nucleotides also enhances general cognitive function, supports healthy stem cell migration and homing, and stem cell renewal. When stem cells reach the site of an injury they must proliferate and expand, as there are not enough migrating stem cells to accomplish full repair of any significant injury or degenerative process. Extracellular nucleotides such as uridine monophosphate (UMP) or a mix of nucleotides such as that found in Nucleoprime™ Nucleotides play a role in this proliferation and expansion by acting as growth factors and supply the building blocks for the RNA and DNA which will be required to build new cells. Clinically, Nucleoprime™ supports faster recovery and promotes improved performance during endurance exercise. See, e.g., Zeng X, Shi C, Han Y, et al. A metabolic atlas of blood cells in young and aged mice identifies uridine as a metabolite to rejuvenate aged hematopoietic stem cells. Nature Aging. 2024; 4 (10): 1477-1492. See also, e.g., Liu Z, Li W, Geng L, et al. Cross-species metabolomic analysis identifies uridine as a potent regeneration promoting factor. Cell Discovery. 2022; 8 (1): 6. For example, as shown in FIG. 7, concentrations of each of cytidine monophosphate (CMP), uridine monophosphate (UMP), guanosine monophosphate e (GMP), and adenosine monophosphate (AMP) comprise approximately 24-26% of the Nucleoprime™ formulation. Nucleoprime™ is produced utilizing a 100% natural fermentation process, is GRAS certified, non-GMO, non-allergenic, vegan & vegetarian friendly and provides clinically proven potency.

Aloe macroclada Leaf Gel Extract (Such as Alomac®)

Aloe macroclada is native to Madagascar, where it has been used by the Malagasy people for centuries to support the natural healing of various conditions, promote rejuvenation, and promote longevity. Aloe macroclada has been shown to enhance stem cell function in the body to a greater degree than any other natural product, and without the toxicity often associated with unnatural products. In a human clinical trial, Aloe macroclada leaf gel extract was shown to enhance hematopoietic stem cells by up to 53%. See, e.g., Drapeau C, Benson K, James J, Jensen G. Aloe macroclada from Madagascar Triggers Transient Bone Marrow Stem Cell Mobilization. Journal of Stem Cell Research and Therapies. 2015; 5. As shown in FIG. 8, for example and not limitation, concentrations of Aloin A+B (1×ppm) in the Alomac® formulation are lower than 3.0 ppm, and polysaccharide content of the Alomac® formulation is approximately 19.2%. Alomac® is halal, kosher, vegan, gluten-free, non-GMO, allergen free, animal free, and both GMP and NSF certified.

### Soluble Beta Glucan (Such as Wellmune® Soluble Baker's Yeast Beta Glucan)

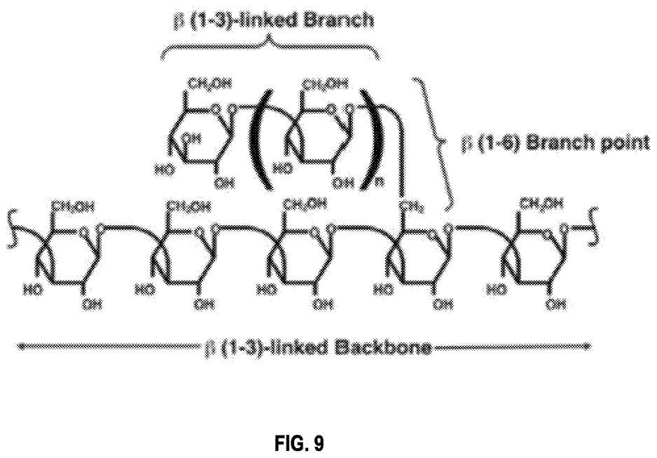

Wellmune® Soluble is a water-soluble baker's yeast beta (β)-1,3/1,6-glucan—a complex polysaccharide found in cell walls of plant-based foods and fungi-known to promote the body's natural defenses. Wellmune® is available in both soluble and dispersible (i.e., particulate) forms and differs from other forms of non-yeast beta-glucan in both chemical structure and biological activity. Specifically, Wellmune® has a backbone of glucose molecules linked together via a beta-(1->3) linkage. In addition, there are branch points along the backbone where side chains are attached via a beta-(1->6) linkage. And each side chain is typically 4-6 glucose units long. See, e.g., FIG. 9. Multiple clinical trials demonstrate the ability of Wellmune® to help strengthen the immune system and promote overall energy, vitality, improved mental clarity, and optimized general immune health and wellness, especially during times of increased stress. β-glucans also support healthy stem cell mobilization, migration, proliferation, and differentiation. See, e.g., Torello C O, Souza-Queiroz J, Queiroz M L. Clin Exp Pharmacol Physiol. 2012; 39 (3): 209-217. See also, e.g., Cramer D E, Allendorf D J, Baran J T, et al. Beta-glucan enhances complement-mediated hematopoietic recovery after bone marrow injury. Blood. 2006; 107 (2): 835-840. β-glucan preparations can be particulate (i.e., dispersible) or soluble. A key difference between particulate and soluble β-glucan is their solubility in water: particulate β-glucan is not readily soluble in water, forming visible particles, while soluble β-glucan dissolves easily in water; this difference in solubility significantly impacts how they interact with the immune system, with particulate β-glucan primarily activating immune cells directly through the Dectin-1 receptor, while soluble β-glucan can bind to complement receptor 3 (CR3) to activate complement system proteins for immune stimulation. This function is important because the complement system is involved in guiding migrating stem cells to where they are needed in the body for tissue regeneration and development. This particulate vs soluble distinction may be especially important for optimizing stem cell functions like migration and proliferation during relatively brief, intermittent dosing protocols, where the intention of the beta glucan is to complement other ingredients that are effective during the days when the combination of ingredients are being taken in order to prevent a time-lag between when the beta glucan and other ingredients are acting. The soluble form of beta glucans can interact with the complement system without further processing by the body, while a dispersible form may require at least 3-4 days to be taken up by gastrointestinal macrophages and transported to the bone marrow, where they are degraded and secreted as small soluble biologically active fragments that can bind to CR3. See Hong F, Yan J, Baran J T, et al. Mechanism by which orally administered beta-1,3-glucans enhance the tumoricidal activity of antitumor monoclonal antibodies in murine tumor models. J Immunol. 2004; 173 (2): 797-806. Wellmune® has broad regulatory approval, including FDA, GRAS, and novel food approval in the EU and China. Wellmune® is kosher, halal, non-allergenic and GMO-free.

### Okinawa Muzuku (Cladosiphon Okamuranus) Extract

Mozuku (Cladosiphon okamuranus) is an edible brown seaweed grown in the coastal waters of Okinawa and is a foundational superfood to this blue zone's diet. This extract contains >=85% fucoidan, a sulfated polysaccharide with numerous health benefits. Fucoidan is a potent compound shown to aid in immune health and to support the healthy mobilization, migration and homing of stem cells. Fucoidan is an L-selectin ligand that may have an effect on stem cell mobilization by up-regulating the chemokine receptor CXCR4, a receptor for SDF-1. See, e.g., Irhimeh M R, Fitton J H, Lowenthal R M. Fucoidan ingestion increases the expression of CXCR4 on human CD34+ cells. Exp Hematol. 2007; 35 (6): 989-994. See also, e.g., Sweeney E A, Lortat-Jacob H, Priestley G V, Nakamoto B, Papayannopoulou T. Sulfated polysaccharides increase plasma levels of SDF-1 in monkeys and mice: involvement in mobilization of stem/progenitor cells. Blood. 2002; 99 (1): 44-51. While this embodiment contemplates Mozuku (Cladosiphon okamuranus) as the source of fucoidan from brown seaweed, other brown seaweeds including but not limited to Fucus spp, Ascophyllum nodosum, Laminaria spp, Sargassum spp, and Undaria pinnatifida may also serve a similar function in the formula.

### Chlorella Growth Factor (C.G.F.)

C.G.F. extract is a hot water extract of chlorella. It takes several grams of chlorella to make a couple hundred milligrams of C.G.F. extract. C.G.F. extract supports healthy growth and healing processes, with nucleic acids, peptides, water-soluble vitamins, and minerals. C.G.F. extract is vegan, non-GMO, gluten-free, encourages healthy stem cell proliferation, supports the immune system, and innate tissue regeneration.

Spirulina Water Extract (such as CyaninPlus®)

Spirulina extracts are made by concentrating and extracting spirulina using water, ethanol, or other extraction methods. A water extract of spirulina has the advantage of concentrating water-soluble compounds such as the blue protein phycocyanin. Derived from Spirulina using a proprietary aqueous extraction method, CyaninPlus® supports a healthy inflammation response through a 5-pronged mechanism of action. Because it is clinically shown to help relieve physical discomfort, CyaninPlus® helps consumers feel better, do more, and enjoy a better quality of life. CyaninPlus®, a brilliant blue extract, contains both phycocyanin (PC) and non-PC compounds—a concentrated combination not seen in standard Spirulina powder or phycocyanin—which contributes to its unique efficacy. CyaninPlus® has been the subject of original in vitro and human clinical research showing multiple benefits, including but not limited to supporting healthy inflammatory responses, promoting healthy joints, helping relieve general discomfort, providing greater physical comfort at rest and during everyday activities, and providing antioxidant and cellular protection and support. CyaninPlus® is extracted through a proprietary aqueous extraction method that concentrates the bioactive constituents without the use of chemical solvents or buffers, derived from natural sources, free of synthetic additives, kosher, halal, FDA certified, and GRAS certified.

### Astragalus Root Extract

Astragalus (Astragalus membranaceus) root extract comprises approximately 5% Astragaloside IV. Astragalosides support healthy stem cell proliferation and differentiation, through various mechanisms including Notch signaling, a highly conserved mechanism from invertebrates to mammals known for determining cell fate. Astragalus root supports wound healing as well as healthy blood circulation by enhancing VEGF, a protein crucial for angiogenesis, cell movement, and tissue repair. Astragalus root extract is vegan, gluten-free, and non-GMO. While the above described Astragalus extract contemplates use of a root extract standardized for Astraloside IV, a root extract standardized for astragalus polysaccharides, or a combination of astragaloside and polysaccharide may also serve a similar function in the formula.

### Notoginseng Root Extract

Notoginseng root extract, or Panax Notoginseng Root Extract, also known as Tienchi ginseng, comprises approximately 5% notoginsenosides. Notoginseng root extract supports healthy stem cell proliferation and differentiation, primarily through the Wnt/β-catenin signaling pathway, which also regulates stem cell self-renewal. Notoginseng root extract also supports healthy blood circulation, promotes a positive response from exosomes supporting a healthy balance of cytokines & immunity, and encourages innate tissue repair. Notoginseng root extract is vegan, gluten-free, non-GMO, kosher and halal. While the above-described extract contemplates use of a root extract standardized for 5% notoginsenosides, a root extract standardized for higher or lower concentrations, with the amount of root extract varied accordingly, may also serve a similar function in the formula.

Salvia miltiorrhiza Root Extract

Salvia miltiorrhiza, also known as Chinese sage, Root Extract supports healthy stem cell proliferation and differentiation through both the Wnt/β-catenin and Notch signaling pathways. Salvia miltiorrhiza influences exosomes and cellular communication to promote cytokine balance and stem cell activity, and contains exosome-like molecules that support wound healing, angiogenesis, and cell migration. Salvia miltiorrhiza Root Extract is vegan, gluten-free, and non-GMO. Extracts concentrated based on extraction ratios (e.g., 8:1, 10:1) or standardized for Salvianolic acid, Tanshinones, or their combination serve a similar function in the formula.

White Peony Root Extract (Paeonia lactiflora)

White peony, or Chinese peony, root extract supports healthy stem cell proliferation, differentiation, renewal, supports innate tissue regeneration, and helps maintain balanced activity of CDC42, which is an important mechanism for epithelial, hematopoietic, intestinal, mesenchymal, and neural stem cell function and/or regeneration. White peony root extract is vegan, vegetarian, allergen-free, gluten-free, non-GMO, BSE- and TSE-free, kosher and halal. Extracts concentrated based on extraction ratios (e.g., 8:1, 10:1) or standardized for paeoniflorin serve a similar function in the formula.

### Ferulic Acid

Ferulic acid is derived from Oryza sativa and is the primary bioactive in Angelica sinensis attributed to its therapeutic properties. Ferulic acid supports healthy stem cell proliferation, differentiation, self-renewal, and survival of specialized cell types. Ferulic acid is perhaps best known for its ability to support wound healing, healthy blood circulation, and innate tissue regeneration through its positive influence on VEGF. Ferulic acid is vegan, gluten-free, non-GMO, and kosher.

### Chlorophyllin (as Sodium Copper or Sodium Magnesium Chlorophyllin)

Sodium copper chlorophyllin supports healthy stem cell proliferation and differentiation and the health of the blood and immune systems by activating relevant stem cells. One of the ways it does this is by enhancing granulocyte-colony stimulating factor (G-CSF) production, which helps mobilize stem cells from bone marrow to peripheral blood. Chlorophyllin is also known for its antioxidant properties, protecting DNA from oxidative damage and potentially reducing genomic instability, a hallmark of aging. Sodium copper chlorophyllin is vegan, vegetarian, allergen-free, gluten-free, non-GMO, BSE- and TSE-free, and halal.

### L-Serine

L-Serine is an amino acid that supports healthy stem cell proliferation, regeneration, and survival mechanisms. Serine plays a crucial role in stem cell maintenance and function by acting as a key metabolite in the “serine-glycine one-carbon pathway,” providing one-carbon units necessary for the synthesis of nucleotides, which are essential for rapid cell proliferation and DNA replication, a critical aspect of stem cell behavior; essentially, exogenous serine availability is vital for stem cells to maintain their self-renewal capacity and differentiate into specialized cell types when needed. L-Serine is vegan, vegetarian, allergen-free, gluten-free, non-GMO, BSE- and TSE-free, and halal.

### Royal Jelly Powder

In some embodiments, royal jelly powder is standardized to comprise 4% 10-hydroxy-2-decenoic acid (10-HDA). Royal jelly powder supports healthy stem cell proliferation, differentiation, innate tissue regeneration, combats cellular senescence, and supports telomere and mitochondrial health. Royal jelly also improves health span, extends lifespan, and enhances fitness with aging. It supports muscle health and stem cells, promoting both muscle stem cell proliferation and the differentiation of neural stem cells into brain cells. Royal Jelly also contains extracellular vesicles which are exosome-like compounds: Exosomes are small vesicles released by cells, including stem cells, that carry proteins, lipids, and nucleic acids. They are a key part of cell-to-cell communication. The exosome-like vesicles in royal jelly are known to interact with stem cells, promote their migration, and enhance wound healing. Royal jelly powder is allergen-free, gluten-free, non-GMO, and BSE- and TSE-free.

### Vitamin C

Vitamin C supports healthy stem cell proliferation, differentiation, and facilitates healthy stem cell survival mechanisms. Vitamin C is vegan, gluten-free, and non-GMO.

### Echinacea Purpurea

Echinacea purpurea extract is standardized to comprise 4% phenolics. Echinacea purpurea supports healthy stem cell migration and homing, healthy formation of new blood vessels, and encourages innate tissue regeneration. Echinacea purpurea is vegan, vegetarian, allergen-free, gluten-free, and non-GMO. Herb, root, or their combination (i.e., whole plant) E. purpurea, and extracts concentrated based on extraction ratios (e.g., 8:1, 10:1) or standardized for E. purpurea serve a similar function in the formula.

### Bromelain

Bromelain supports healthy stem cell proliferation, differentiation, and encourages innate tissue regeneration. A key factor that allows stem cells to function effectively is a healthy microenvironment-which includes balanced immunity and inflammation, access to signaling molecules, nutrients, and supportive cells, and proper regulatory mechanisms. Bromelain's proteolytic activity is thought to facilitate the removal of damaged tissue and support a balanced inflammatory and immune response, thereby creating a more favorable microenvironment for stem cell activities and tissue regeneration.

Combining Composition 100 with Other Products & Protocols

The present disclosure also contemplates combining composition 100 with one or more other products and/or compositions where particular health and/or physiological outcomes are desired. Examples of such other products and/or compositions may include, but are not limited to: Qualia Senolytic®, which is formulated to combat cellular senescence; Qualia Mitochondria+, which is formulated to reduce mitochondrial dysfunction; Qualia Joint Health™, which is formulated to reduce stiffness and aches in joints; Qualia Probiotic+i.e., Qualia Synbiotic®, which is formulated to support gastrointestinal health; Qualia Mind® and Qualia Mind® Caffeine free which is formulated to support brain and cognitive health; and Qualia NAD+® and/or Qualia Magnesium+®, which are formulated for general coverage of multiple of the hallmarks of aging (see, e.g., FIG. 1).

Combining Composition 100 with Qualia Senolytic® for Obtaining a Synergistic Health Effect of Body Optimization and/or Physiological Harmony

In some combined embodiments, composition 100 may be combined and/or dosed along with Qualia Senolytic® to obtain a synergistic health effect of body optimization & harmony. For example, cycles of clearing out zombie cells can be deliberately encouraged by taking the Qualia Senolytic® senolytic formula, and growth of new, healthy cells can be promoted by taking composition 100, a stem cell support formula. Such a combination may provide mutual support for one another. For example, Qualia Senolytic® may create an environment of physiological receptivity for composition 100, while composition 100 actively builds new foundations and vitality within the body. Contrarily, composition 100 may create an environment of physiological receptivity for Qualia Senolytic®, while Qualia Senolytic® actively builds new foundations and vitality within the body.

Notably, the senescence-associated secretory phenotype (SASP), which is the combination of cellular signals that promotes cellular senescence, is also suspected to contribute to impaired stem cell renewal. Olive leaf extract in the Qualia Senolytic® may help to dampen the SASP, and in doing so, may also help liberate stem cell renewal.

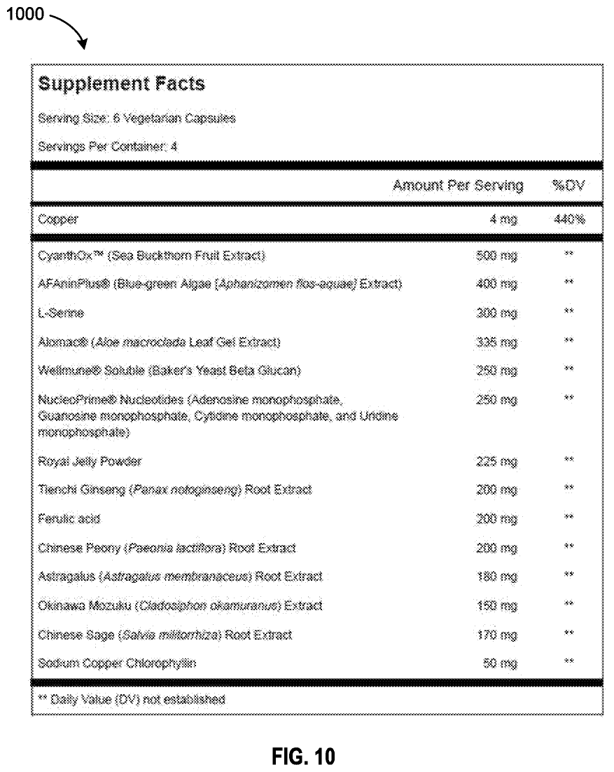

A one month protocol for obtaining a synergistic health effect of body optimization & harmony may include dosing during one week each month, taking 6 capsules of Qualia Senolytic® on Monday and another 6 capsules of Qualia Senolytic® on Tuesday, followed by taking, e.g., 6 capsules of composition 100 (e.g., the formulation described by label 300 of FIG. 3 or label 400 of FIG. 4 or label 1000 of FIG. 10) on Wednesday, 6 capsules on Thursday, 6 capsules on Friday, and 6 capsules on Saturday.

Combining Composition 100 with Qualia NAD+® for Mitigating One or More of the 12 Hallmarks of Aging

Stem cells are susceptible to the same stressors as regular cells, such as the processes ascribed to the 12 hallmarks of aging, like genomic instability, telomere attrition, cellular senescence, mitochondrial depletion, dysregulated nutrient sensing, and stem cell exhaustion. Accordingly, in some other combined embodiments, composition 100 may be combined and/or dosed along with Qualia NAD+® to obtain a synergistic health effect of mitigating several of these hallmarks of aging. For example, NAD+supports stem cell health primarily through its help for mitigating these hallmarks. Qualia NAD+comprises 3 forms of NAD+boosting vitamers of vitamin B3 to support NAD+synthesis: nicotinic acid (NA, also known as niacin), nicotinamide (NAM), and nicotinamide riboside (NR). Proper NAD+ levels help to maintain stem cells' “stemness” (e.g., the ability to self-renew and differentiate) and avoid stem cell exhaustion. Maintenance of NAD+pools also helps to prevent cellular senescence. See, e.g., Navas, L. E., Carnero, A. NAD⁺ metabolism, stemness, the immune response, and cancer. Sig Transduct Target Ther 6, 2 (2021). https://doi.org/10.1038/s41392-020-00354-w. See also, e.g., Son, M. J., Kwon, Y., Son, T. and Cho, Y. S. (2016), Restoration of Mitochondrial NAD+Levels Delays Stem Cell Senescence and Facilitates Reprogramming of Aged Somatic Cells. Stem Cells, 34:2840-2851. https://doi.org/10.1002/stem.2460. Increasing NAD+has been shown to restore homeostasis and mitochondrial fitness in senescent stem cells through a SIRT-1 mediated mechanism. See, e.g., Yuan, X., Liu, Y., Bijonowski, B. M. et al. NAD+/NADH redox alterations reconfigure metabolism and rejuvenate senescent human mesenchymal stem cells in vitro. Commun Biol 3, 774 (2020). https://doi.org/10.1038/s42003-020-01514-y. The main consumers of NAD+ (CD38, PARPs, SIRT) are also known to interact with stem cell mechanisms through numerous hallmarks of aging, such as maintenance of genomic integrity and telomeres. From a bigger picture perspective, there is also evidence that NAD+boosting promotes stem cell functionality in multiple tissues and enhances lifespan. See, e.g., Igarashi M, Miura M, Williams E, Jaksch F, Kadowaki T, Yamauchi T, Guarente L. NAD⁺ supplementation rejuvenates aged gut adult stem cells. Aging Cell. 2019 June; 18 (3): e12935. doi: 10.1111/acel. 12935. Epub 2019 Mar. 27. PMID: 30917412; PMCID: PMC6516145. See also, e.g., Zhang H, Ryu D, Wu Y, Gariani K, Wang X, Luan P, D'Amico D, Ropelle E R, Lutolf M P, Aebersold R, Schoonjans K, Menzies K J, Auwerx J. NAD⁺ repletion improves mitochondrial and stem cell function and enhances life span in mice. Science. 2016 Jun. 17; 352 (6292): 1436-43. doi: 10.1126/science.aaf2693. Epub 2016 Apr. 28. PMID: 27127236.

Interestingly, a recent publication on NR/NMN supplementation in mice revealed that nicotinamide (NAM) supplementation helped promote stem cell activity and muscle regeneration using an NAD+independent mechanism (NAM alone, regardless of NAD+boosting, supports stem cells). See, e.g., Ancel S, Michaud J, Migliavacca E, Jomard C, Fessard A, Garcia P, Karaz S, Raja S, Jacot G E, Desgeorges T, Sánchez-García J L, Tauzin L, Ratinaud Y, Brinon B, Metairon S, Pinero L, Barron D, Blum S, Karagounis L G, Heshmat R, Ostovar A, Farzadfar F, Scionti I, Mounier R, Gondin J, Stuelsatz P, Feige J N. Nicotinamide and pyridoxine stimulate muscle stem cell expansion and enhance regenerative capacity during aging. J Clin Invest. 2024 November 12; 134 (24): e163648. doi: 10.1172/JCI163648. PMID: 39531334; PMCID: PMC11645154.

In such combinations, a one-month protocol for mitigating one or more of the hallmarks of aging may include dosing during one week each month. Specifically, take 6 capsules of composition 100 (e.g., the formulation represented by label 300 in FIG. 3 or label 400 of FIG. 4 or label 1000 of FIG. 10) for 4 consecutive days during the month. Also, take Qualia NAD+® daily. Optionally, a double dose of Qualia NAD+® may be taken for 1 week each month, starting on the first day taking composition 100, and taking 1 week off from taking Qualia NAD+® each month.

Combining Composition 100 with Qualia Joint Health™ for Improving and/or Supporting Joint Health

The primary nature of Qualia Joint Health™ is as a potent anti-inflammatory and to support the structural characteristics of joints, tendons, cartilage, etc. Both of these aspects are synergistic with composition 100. By supporting the healthy regulation of inflammation, and even immune function, Qualia Joint Health™ helps to provide a healthier environment, which facilitates improved recovery and healing and is, overall, synergistic with the intent of composition 100, where supporting a healthy inflammatory balance may also promote improved stem cell signaling and activity. Balanced inflammatory processes support stem cell maintenance throughout the month, and allow stem cells to be involved in generalized healing & rejuvenation when mobilized, rather than being dedicated to addressing inflammation.

Qualia Joint Health™ also comprises ingredients to support the protection, integrity, regeneration, and health of the different structural aspects of the joint. Certain stem cell types fundamentally turn into these structural aspects (ex: cartilage). From this perspective, composition 100 supports and amplifies this aspect of Qualia Joint Health™. In other words, taking Qualia Joint Health™ following composition 100 would support renewal of joint tissues and connective tissues such as tendons. Lastly, the ingredients of Qualia Joint Health™ also promote various aspects of stem cell activity: Curcumin in turmeric, Boswellia, Ginger, L-Carnitine (ALCAR), and Hyaluronic Acid in Ovomet®.

A suggested monthly protocol for improving and/or supporting joint health may include taking 6 capsules of composition 100 (e.g., a formulation according to label 300 of FIG. 3 or label 400 of FIG. 4 or label 1000 of FIG. 10) daily for 4 consecutive days during the month, while taking Qualia Joint Health™ daily throughout the month. Optionally, dosing Qualia Joint Health™ may be abstained from on each of the 4 days during which composition 100 is dosed, and then a double dose of Qualia Joint Health™ may be taken on each of the first few days following dosing of composition 100.

Combining Composition 100 with Qualia Joint Health™ for Improving and/or Supporting Sports Recovery

Composition 100 encourages healthy stem cell activity and promotes tissue healing and rejuvenation, which also has applications for athletes looking to optimize recovery after a workout, especially muscles & other tissues, or looking for support to heal from short term and/or long-term injuries, sports related or otherwise. While relevant for people of all ages, this is particularly relevant for aging populations, since older athletes must prioritize recovery if they want to maintain muscle mass and/or athletic ability, prevent injury, and/or continue making gains.

A suggested monthly protocol for improving and/or supporting sports recovery may include taking 6 capsules of composition 100 (e.g., a formulation according to label 300 of FIG. 3 or label 400 of FIG. 4 or label 1000 of FIG. 10) daily for 4 consecutive days during the month. Time these doses to follow a period of intense exercise to support recovery (e.g., an event, a more intense training week, etc). Also, dose Qualia Joint Health™ daily throughout the month. Optionally, dosing Qualia Joint Health™ may be abstained from on each of the 4 days during which composition 100 is dosed, and then a double dose of Qualia Joint Health™ may be taken on each of the first few days following dosing of composition 100.

Combining Composition 100 with Qualia Probiotic+for Improving and/or Supporting GI Renewal

Intestinal stem cells divide continuously throughout life to produce the cells lining the surface of the small and large intestines. The constant self-renewal and differentiation of adult intestinal stem cells maintains a functional intestinal mucosa for a lifetime. This allows for combined dosing of composition 100 and Qualia Probiotic+ to improve and/or support GI cellular renewal.

A suggested monthly protocol for improving and/or supporting GI renewal may include taking 6 capsules of composition 100 (e.g., a formulation according to label 300 of FIG. 3 or label 400 of FIG. 4 or label 1000 of FIG. 10) daily for 4 consecutive days during the month, while dosing Qualia Probiotic+daily throughout the month.

Combining Composition 100 with Qualia Mind® or Qualia Mind® Caffeine Free for Improving and/or Supporting Brain Performance

While neurogenesis is limited in adults, to at most the hypothalamus and olfactory brain regions, neuroplasticity is not so limited, and other brain cells besides neurons are capable of cellular renewal. The combination of Qualia Mind® or Mind Caffeine Free) plus composition 100 supports the body's innate ability to make new brain cells and experience neuroplasticity, while gaining the second-to-none cognitive benefits of Qualia Mind®.

A suggested monthly protocol for improving and/or supporting brain performance, neuroplasticity, and/or rejuvenation may include taking 6 capsules of composition 100 (e.g., a formulation according to label 300 of FIG. 3 or label 400 of FIG. 4 or label 1000 of FIG. 10) daily for 4 consecutive days during the month, while dosing Qualia Mind® (or Mind Caffeine Free) 5 days each week of the month.

Combining Composition 100 with Qualia Mind® or Qualia Mind® Caffeine Free for Improving and/or Supporting Generalized Wound Healing

It is a common stereotype that wound healing is reduced & delayed with aging. However, a clinical study noted that older women exhibited 95% larger wounds than younger men 5 days after wounding (a mucosal wound). See, e.g., Engeland C G, Bosch J A, Cacioppo J T, Marucha P T. Mucosal Wound Healing: The Roles of Age and Sex. Arch Surg. 2006; 141 (12): 1193-1197. doi: 10.1001/archsurg.141.12.1193. This observation is also theoretically applicable to muscle, bone, skin, and immunity during the healing process. As described above, composition 100 comprises compounds and botanicals that help reinforce healing and rejuvenation across numerous cell types and organ systems in animal models. Encouraging stem cell activity at regular intervals may help to maintain youthful tissue healing/repair over time, and with aging.

A suggested monthly protocol for improving and/or supporting generalized wound healing support may include taking 6 capsules of composition 100 (e.g., a formulation according to label 300 of FIG. 3 or label 400 of FIG. 4 or label 1000 of FIG. 10) daily for 4 consecutive days during the month to also support healing potential.

### Scientific Studies on Efficacy of Composition(s) 100 as Described Herein

Compound 100 is configured to help promote stem cells functions including mobilization, migration, proliferation, and differentiation, as well as stem cell maintenance functions. This is intended to help combat the aging process, help activate cellular longevity processes, and help repair and rejuvenate tissues.

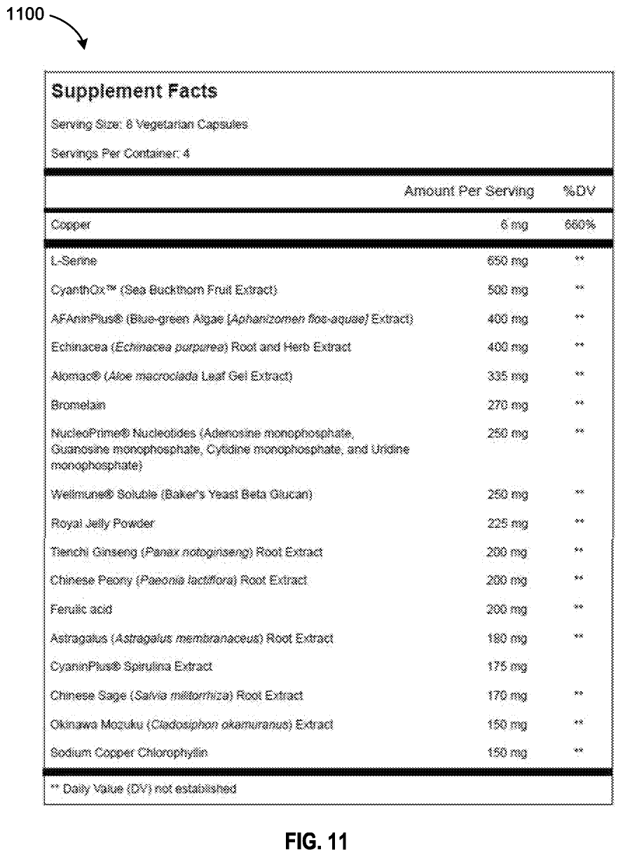

In a first pilot clinical trial of 2 versions (a Version A, as shown in the label 1000 of FIG. 10, and a Version B, as shown in label 1100 of FIG. 11) of a stem cell formula was carried out with 29 healthy individuals aged 40 to 75 years old (14 males and 15 females) between February 2025 and March 2025. Version A was a 6-capsule formula and version B was an 8-capsule formula. Participants took 6 or 8 capsules of A or B, respectively, for 4 consecutive days.

Male and female symptoms of aging were evaluated using the Aging Male Symptom scale (AMS) and a version of this scale designed for females (AFS). The symptoms of aging scores decreased in males by ~30% with similar changes in both the 6 and 8 capsule groups, while the scores decreased in females more in the 6-capsule group (~42%) compared to the 8capsule group (~14%).

Participants were also asked to respond to questions about healing and well-being. Overall, 19% reported tissue healing or rejuvenation, 33% noticed energy changes or improved well-being, 27% noticed improvements in joint/muscle aches & pains, 30% noticed improvements in focus/mental clarity, and 7% noticed improvements in skin or nails. Given the short duration of the study, it was interesting to find that 44% of participants noticed a benefit in 1 or more of these areas.

Given the brief duration of this study, both versions worked unexpectedly well for a range of symptoms related to aging that encompassed physical, psychological, and sexual symptoms, and elicited improvements in a variety of areas related to tissue healing and rejuvenation.

A second, proteomic pilot study was conducted to investigate the mechanistic basis by which compositions(s) 100 may support stem cell activity. Proteomics is a method of analyzing proteins produced by cells and reflects how cells are responding in real time, and how genetic information is being translated into cellular action. This study examined numerous cytokines, or proteins that act as chemical messengers, using the Olink Target 48 Cytokine panel. Approximately a dozen of the cytokines measured were more strongly implicated in stem cell biology.

In this second, proteomic study, six participants aged 50-67 years (5 males, 1 female) completed a blood spot test to assess baseline levels of selected circulating proteins. Participants consumed composition 100, in either a 6-capsule formula as shown in FIG. 10 or a 8-capsule formula as shown in FIG. 11, according to the package directions on each of days 1 through 4. Following 4 days of supplementation, participants completed a second blood spot test to assess circulating protein levels after supplementation.

In the oldest participant (aged 67), who took the 6 capsule formula, circulating levels of granulocyte-macrophage colony-stimulating factor (GM-CSF) increased by 770% compared to baseline, representing one of the largest single responses observed in the entire study. GM-CSF was one of the primary stem cell mechanisms targeted by the ingredients in composition 100, and is known to support stem cell migration and differentiation.

Two participants, both of whom took the 6 capsule formula shown in FIG. 10, demonstrated increased levels of CXC motif chemokine 12 (CXCL12, also known as SDF-1), a cytokine involved in the CXCR4/SDF-1 pathway, which was another primary stem cell mechanism targeted by ingredients in composition 100. Increases in CXCL12 suggest enhanced capacity for stem cells to mobilize from the bone marrow and migrate to target tissues. These two participants' CXCL12 levels were increased by 132% and 324%, respectively. Of note, Panax notoginseng, fucoidan, and Aphanizomenon flos-aquae (AFA) have demonstrated prior evidence of modulating the CXCR4/SDF-1 pathway. Fucoidan and AFA supplementation have also increased stem cell numbers in prior human clinical trials.

The largest individual effect observed in the proteomics study was in one participant who took the 8 capsule formula as shown in FIG. 11, whose interleukin-10 (IL-10) levels increased by 9,300% compared to baseline. IL-10 is an anti-inflammatory cytokine that promotes healthy inflammatory processes and can directly stimulate stem cell self-renewal and regeneration.

This study also examined overall cytokine “signature” or “profile” changes for each individual comparing baseline to post-supplementation levels. In the oldest participant (aged 67 taking the 6 capsule formula of FIG. 10), the most notable cytokine changes included: a 2,872% increase in IL-17F, a 903% increase in IL-2, a 770% increase in GM-CSF, a 387% increase in IL-27, a 202% increase in IL-10, a 143% increase in IL-7, a 132% increase in CXCL12, a 121% increase in IFN-γ, a 21% decrease in HGF, and 15% decrease in FLT3LG. The overall pattern in this participant including rising CSF2, increased CXCL12, and normalization of growth factor signaling including HGF and FLT3LG, is physiologically consistent with a productive “mobilize->home→stabilize” sequence, which may reflect healthy regenerative activation rather than chronic inflammation.

In yet another case study, the cytokine signature of a participant aged 59 taking the 6 capsule formula shown in FIG. 10 suggested a regenerative activation phenotype, with a short burst of innate activation (reflected by increases in IL-1β and IL-33) that may support progenitor signaling and matrix turnover, alongside changes in EGF, VEGFA, IL-13, and IL-2 consistent with enhanced tissue healing and immune coordination. The pattern of CXCL12 changes in this participant may additionally reflect transient mobilization of stem cells into circulation.

Several additional cytokines met the threshold for meaningful fold-change related to baseline, including matrix metalloproteinase-12 (MMP12), CXC motif chemokine ligand 8 (CXCL8), and interleukin-1 beta (IL-1B), which all decreased compared to baseline. MMP12 is primarily involved in breaking down and remodeling the extracellular matrix as part of tissue repair and wound healing. CXCL8 is a chemoattractant for immune and stem cells. IL-1B is a proinflammatory mediator that can function to either promote or inhibit stem cell activity depending on context. Taken together, these reductions may reflect a normalization of inflammatory and tissue-remodeling signals consistent with the broader regenerative and repair-dominant profile observed across participants. Composition 100 works unexpectedly well in that, by using multiple ingredients that support a variety of functions of and types of stem cells, the composition has a more robust effect than the individual plant extracts on their own. The constituents form a synergistic compound where the combined effect is greater than the sum of their separate effects. The intermittent dosing balances effectiveness and safety.

### Method of Use

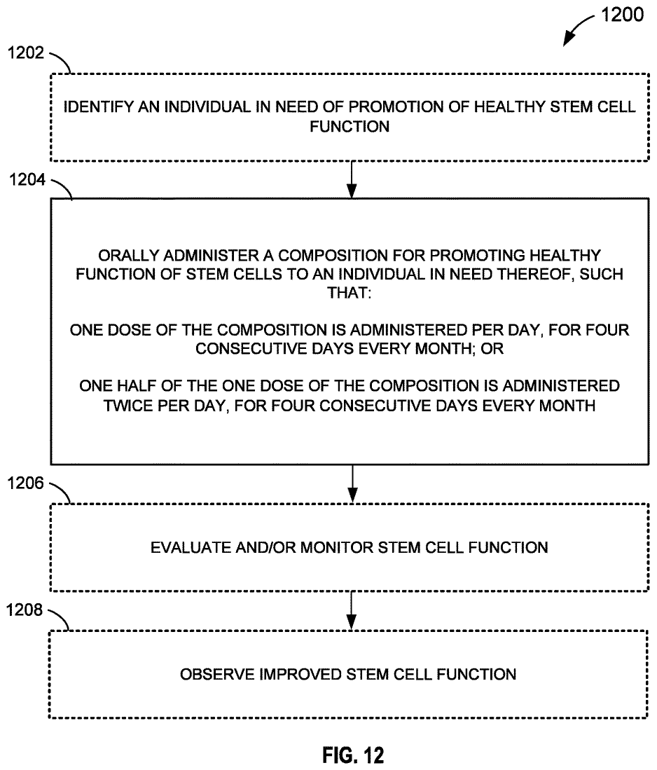

The disclosure now turns to FIG. 12, which illustrates a flowchart 1200 related to a method of supporting stem cell health as described anywhere in this disclosure. Although particular steps are described herein, the present application is not so limited and alternative methods may include a subset of these steps, in the same or different order, and may additionally include one or more additional steps not described herein. The compositions and methods described herein support the body's natural regenerative processes, promote healthy aging, and encourage normal tissue repair.

In some embodiments, flowchart 1200 includes a step 1202, which includes identifying an individual in need of promotion of healthy stem cell function. Such identification may be made in any number of ways. Individuals in need of activation of stem cells can include, without limitation, individuals presenting with tissue degeneration, slower repair capacity, high physical demand, chronic inflammatory burden, age-related decline, or combinations thereof. Because natural stem cell function declines with age both in terms of number of stem cells and stem cell responsiveness, at least one class of individuals can include aging adults, i.e. individuals over forty years old, who can benefit from regenerative health in response to conditions and challenges of aging. Through stem cell activation, the compositions described herein can improve skin integrity and elasticity, promote muscle mass maintenance, address bone density, maintain joint health, and encourage wound healing. In another aspect, the disclosed methods support individuals with musculoskeletal stress or injury including, without limitation, athletes, post-surgical patients (including orthopedic surgery patients, spinal surgery patients, and joint replacement patients, for example, as well as individuals in need of wound repair), individuals with chronic joint pain such as those presenting with osteoarthritis, and individuals recovering from tendon and/or ligament injuries, wherein the promotion of healthy stem cell function can enhance bone, cartilage, tendon, and muscle tissue repair. Individuals in need of promotion of healthy stem cells can also include individuals with degenerative conditions involving tissue deterioration such as those presenting with osteoarthritis, intervertebral disc degeneration, sarcopenia, osteoporosis, as well as neurodegenerative conditions associated with aging. An individual in need of stem cell promotion can likewise include an individual presenting with chronic inflammation. The chronic inflammation can be the result of metabolic syndrome, diabetes, autoimmune conditions, obesity, and the like. In still another aspect, an individual in need of healthy stem cell promotion can be a high performance/bio-optimization individual with an interest in longevity and wellness.

Many of the same core mechanisms recur and define additional contexts likely to benefit from stem-cell-targeted strategies. These include but are not limited to (1) chronic oxidative stress and accumulation of toxic metabolites (reactive oxygen species [ROS]-driven DNA, protein, and lipid damage), (2) mitochondrial dysfunction and impaired mitophagy in stem and niche cells, (3) genomic instability, telomere erosion, and DNA repair deficits, (4) epigenetic remodeling (global and locus-specific DNA methylation changes, histone modifications) that shift stem cell fate and exhaustion risk, (5) loss of quiescence or proliferative exhaustion of stem/progenitor pools, (6) senescence and SASP in stem and niche cells, with elevated TGF-β, IL-6, IL-8, TNF-α, etc., impairing regeneration, and (7) niche deterioration: inflammatory, fibrotic, or metabolically deranged microenvironments failing to provide appropriate protective and instructive cues.

Any clinical or subclinical state, as well as persons impacted by a range of diet, lifestyle and environmental factors that prominently features these mechanisms is a plausible target for interventions designed to preserve or restore stem cell function. Additional clinical or subclinical states may include, but are not limited to cardiovascular disease, osteoporosis, neurodegenerative diseases, and autoimmune and chronic inflammatory diseases. Dietary patterns and exposures that tend to worsen classical hallmarks of aging and stem cell exhaustion correlate with impaired stem cell function. These can include chronic caloric surplus and “Western” diet (high sugar, high saturated fat, ultra-processed foods) or other dietary patterns that promote insulin resistance, ectopic lipids, and metabolic inflammation. Nutrient deficiencies relevant to one-carbon metabolism and redox balance (e.g., methionine/SAM cycle disturbances, impaired antioxidant defenses) can alter epigenetic state and cause ROS damage in stem cells. Excessive alcohol intake causes oxidative stress, mitochondrial damage, inflammatory shifts in several stem cell compartments. Persons with 1 or more lifestyle factors including physical inactivity and sedentary behavior, smoking, chronic psychological stress, poor sleep, and circadian disruption (shift work, irregular light exposure) can benefit because these factors can accelerate aging, worsen insulin resistance and adiposity, reduces progenitor cell regenerative signaling, create ROS, DNA damage, vascular stem/progenitor injury, and contribute to a pro-inflammatory milieu and hormonal patterns that can disturb stem cell niches. Environmental toxicants that drive ROS, inflammation, and epigenetic changes commonly impair hematopoietic and mesenchymal stem cell survival and function. Individuals with high air pollution or occupational toxicant exposure are also strong candidates for interventions aiming to protect or restore stem cell compartments and niches.

Step 1204 includes orally administering a composition 100 to an individual in need thereof, as described anywhere in this disclosure. For example, and not limitation, one of the doses is administered per day, for four consecutive days every month, or one half of the dose is administered twice per day, for four consecutive days every month.

In some embodiments, a dose of such a composition 100 may comprise the ingredients in amounts as shown on label 300 in FIG. 3. In some embodiments, a dose of such a composition 100 may comprise the ingredients in amounts as shown on label 400 in FIG. 4. In some embodiments, a dose of such a composition 100 may comprise a combination of the ingredients in the amounts as shown on label 500 in FIG. 5. In some embodiments, a dose of such a composition 100 may comprise a combination of the ingredients in amounts as shown in TABLE 1. In some embodiments, a dose of such a composition 100 may comprise a combination of the ingredients in amounts as shown in TABLE 2. In some embodiments, a dose of such a composition 100 may comprise a combination of the ingredients in amounts as shown in TABLE 3. In some embodiments, a dose of such a composition 100 may comprise a combination of the ingredients in amounts as shown in TABLE 4. In some embodiments, a dose of such a composition 100 may comprise a combination of the ingredients in the amounts as shown in FIG. 10. In some embodiments, a dose of such a composition 100 may comprise a combination of the ingredients in the amounts as shown in FIG. 11. In some such embodiments, the composition 100 is formulated to be taken orally: one of the dose per day, for four consecutive days every month; or one half of the dose twice a day, for four consecutive days every month. However, the present disclosure is not so limited and composition 100 may be dosed in any manner discussed anywhere in this disclosure.

In some embodiments, flowchart 1200 includes a step 1206, which includes evaluating and/or monitoring stem cell function of the dosed individual. For example, following oral ingestion of compounds targeting improved stem cell function, a cascade of events occur, starting with activation of epithelial and immune cells residing in the gut and vagus nerve signaling, followed by the occurrence of systemic changes to cytokine levels and changes to stem cell trafficking (enhanced surveillance). A direct quantitative method of assessing stem cell mobilization and migration can be evaluated through monitoring of the numbers of various types of stem cells in the blood circulation. Depending on the markers used, one or more of endothelial stem cells, mesenchymal stem cells, pluripotential stem cells, progenitor cells, endothelial stem cells, lymphocytoid mesenchymal-like stem cells, and very small embryonic-like stem cells (VSELS) can be quantified. Another method of monitoring stem cell function is to measure proteins in the blood circulation—specific cytokines, chemokines, and restorative growth factors—such as those described in paras. [0143]-[0150]—that show in real time how signaling factors are impacting stem cell functions, including mobilization, migration, proliferation and differentiation. Qualitative measures can also be used to assess response to interventions targeting stem cell function. Stem cell exhaustion, as defined within the Hallmarks of Aging framework (see López-Otín C, Blasco M A, Partridge L, Serrano M, Kroemer G. Hallmarks of aging: An expanding universe. Cell. 2023 Jan. 19; 186 (2): 243-278. doi: 10.1016/j.cell.2022.11.001. Epub 2023 Jan. 3. PMID: 36599349) is not based on quantitative parameters but rather a recognition that decline in regenerative potential of tissues, which manifests in qualitative issues such as reduced tissue renewal and impaired tissue repair upon injury, is one of the most obvious characteristics of aging. Because doctors do not directly “see” stem cells, historically they have inferred improved stem cell function from symptom patterns, healing trajectories, and performance changes that align with better tissue repair, vascular function, and reduced inflammatory burden. Subjective symptom assessments such as those described anywhere herein, are an alternative method of gauging response to stem cell interventions.

In some embodiments, flowchart 1200 includes a step 1208, which includes observing improved stem cell function in the dosed individual. For example, following oral ingestion of composition 100, as described anywherein herein, targeting improved stem cell function, there are a variety of objective and subjective methods for observing improved stem cell function in a dosed individual. These can include observing objective increases in the various types of stem cells in the blood circulation (e.g., endothelial stem cells, mesenchymal stem cells, pluripotential stem cells, progenitor cells, endothelial stem cells, lymphocytoid mesenchymal-like stem cells) or quantifying alterations of specific cytokines, chemokines, and restorative growth factors that show in real time how signaling factors are impacting stem cell functions, including mobilization, migration, proliferation and differentiation. Subjective experiences reported by the patient or observed by the physician have historically been the primary approach used as evidence that a stem-cell-targeted intervention is having meaningful biological effects. These symptoms include improvements in tissue healing, faster or better-quality recovery from tissue injury, less musculoskeletal pain and better joint comfort, reduced fatigue, more stamina and energy, greater feeling of well-being, improved exercise capacity and physical function, enhanced cognitive clarity and mental performance, better mood, emotional resilience, overall quality of life and daily functioning. A variety of validated research instruments (e.g., SF-36, WOMAC, Frailty Indexes, MMSE) and functional tests (e.g., handgrip strength, gait speed) are used in clinical and research settings to assess these symptoms.

### Method of Manufacture

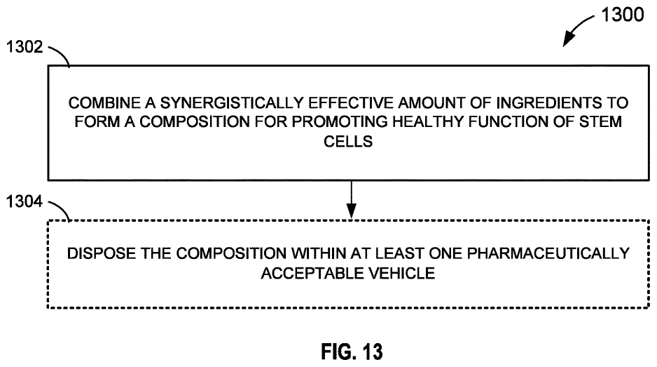

The disclosure now turns to FIG. 13, which illustrates a flowchart 1300 related to a method of manufacturing a composition for supporting stem cell health as described anywhere in this disclosure. Although particular steps are described herein, the present application is not so limited and alternative methods may include a subset of these steps, in the same or different order, and may additionally include one or more additional steps not described herein.

Step 1302 includes combining a synergistically effective amount of ingredients as disclosed anywhere in this disclosure. For example, and not limitation, a dose of such a composition 100, once so-manufactured, may comprise a combination of the ingredients in amounts as shown on label 300 in FIG. 3. In some embodiments, a dose of such a composition 100, once so-manufactured, may comprise a combination of the ingredients in amounts as shown on label 400 in FIG. 4. In some embodiments, a dose of such a composition 100, once so-manufactured, may comprise a combination of the ingredients in the amounts as shown on label 500 in FIG. 5. In some embodiments, a dose of such a composition 100, once so-manufactured, may comprise a combination of the ingredients in amounts as shown in TABLE 1. In some embodiments, a dose of such a composition 100, once so-manufactured, may comprise a combination of the ingredients in amounts as shown in TABLE 2. In some embodiments, a dose of such a composition 100, once so-manufactured, may comprise a combination of the ingredients in amounts as shown in TABLE 3. In some embodiments, a dose of such a composition 100, once so-manufactured, may comprise a combination of the ingredients in amounts as shown in TABLE 4. In some embodiments, a dose of such a composition 100 may comprise a combination of the ingredients in the amounts as shown in FIG. 10. In some other embodiments, a dose of such a composition 100 may comprise a combination of the ingredients in the amounts as shown in FIG. 11. In some such embodiments, the composition 100 is formulated and/or manufactured to be taken orally, for example, one of the doses per day, for four consecutive days every month; or, alternatively, one half of the dose twice per day, for four consecutive days every month. However, the present disclosure is not so limited and composition 100 may be dosed in any manner discussed anywhere in this disclosure.

In some embodiments, flowchart 1300 further includes a step 1304, which includes disposing the composition within at least one pharmaceutically acceptable vehicle 102. In some embodiments, the pharmaceutically acceptable vehicle is an oral capsule 102 (see, e.g., FIG. 1).

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical, or physical partitioning and configurations can be implemented to implement the desired features of the present disclosure. Also, a multitude of different constituent module names other than those depicted herein can be applied to the various partitions. Additionally, with regard to diagrams, operational descriptions, and method claims, the order in which the steps are presented herein shall not mandate that various embodiments be implemented to perform the recited functionality in the same order unless the context dictates otherwise.

Although the disclosure is described above in terms of various embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “typical,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “element” does not imply that the components or functionality described or claimed as part of the element are all configured in a common package. Indeed, any or all of the various components of an element can be combined in a single package or separately maintained and can further be distributed in multiple groupings or packages or across multiple locations.

Additionally, the various embodiments set forth herein are described in terms of exemplary diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

1. A composition for supporting stem cell health in humans, a dose of the composition comprising a synergistically effective amount of:
at least one of:
sea buckthorn fruit extract,
blue-green algae extract, and
Aloe macroclada leaf extract;
soluble beta glucan;
Panax notoginseng root extract;
royal jelly powder;
L-serine;
okinawa mozuku extract; and
nucleotides.

2. The composition of claim 1, the dose of the composition comprising a synergistically effective amount of:
sea buckthorn fruit extract;
blue-green algae extract;
Aloe macroclada leaf extract;
L-serine;
soluble beta glucan;
okinawa mozuku extract;
nucleotides;
royal jelly powder;
ferulic acid;
Panax notoginseng root extract;
white peony root extract;
Salvia miltiorrhiza root extract;
astragalus root extract; and
a cholorophyllin compound.

3. The composition of claim 1, wherein:
the sea buckthorn fruit extract comprises Hippophaeo rhamnoides extract;
the blue-green algae extract comprises Aphanizomenon flos-aquae extract;
the soluble beta glucan comprises baker's yeast soluble beta-1,3/1,6-glucan;
the okinawa mozuku extract comprises Cladosiphon okamuranus extract;
the nucleotides comprise:
uridine monophosphate, or
cytidine monophosphate,
uridine monophosphate,
guanosine monophosphate, and
adenosine monophosphate;
the white peony root extract comprises Paeonia lactiflora root extract;
the astragalus root extract comprises Astragalus membranaceus extract; and
the chlorophyllin compound comprises sodium copper chlorophyllin.

4. The composition of claim 2, wherein the synergistically effective amount comprises:
approximately 250-700 mg of the sea buckthorn fruit extract (500 mg);
approximately 300-1500 mg of the blue-green algae extract (400 mg);
approximately 200-400 mg of the Aloe macroclada leaf extract (335 mg);
approximately 300-3000 mg of the L-serine (300 mg);
approximately 50-300 mg of the soluble beta glucan (250 mg);
approximately 100-1500 mg of the okinawa mozuku extract (150 mg);
approximately 100-500 mg of the nucleotides (250 mg);
approximately 100-500 mg of the royal jelly powder (225 mg);
approximately 100-300 mg of the ferulic acid (200 mg);
approximately 50-400 mg of the Panax notoginseng root extract (200 mg);
approximately 100-500 mg of the white peony root extract (200 mg);
approximately 100-500 mg of the salvia miltiorrhiza root extract (170 mg);
approximately 50-1000 mg of the astragalus root extract (180 mg); and
approximately 25-300 mg of the sodium copper cholorophyllin (50 mg).

5. The composition of claim 2, wherein the synergistically effective amount comprises approximately 50-500 mg of bromelain (270 mg).

6. The composition of claim 3, wherein:
the sea buckthorn fruit extract comprises approximately 150 mg of sea buckthorn proanthocyanidins;
the nucleotides comprise:
approximately 24-26% cytidine monophosphate,
approximately 24-26% uridine monophosphate,
approximately 24-26% guanosine monophosphate, and
approximately 24-26% adenosine monophosphate;
the royal jelly comprises 4% 10-hydroxy-2-decenoic acid (10-HDA);
the white peony extract comprises 50% paeoniflorin;
the astragalus membraneacous root extract comprises approximately 5% astragalosides;
the okinawa mozuku extract comprises at least 85% fucoidan; and
the Panax notoginseng root extract comprises approximately 5% Notoginsenosides.

7. The composition of claim 5, wherein the synergistically effective amounts of the ferulic acid, the astragalus root extract, the Panax notoginseng root extract, the salvia root extract, the white peony root extract and the okinawa mozuku extract are formulated to support healthy blood circulation.

8. The composition of claim 5, wherein the synergistically effective amounts of the sea buckthorn fruit extract, the blue-green algae extract, the Aloe macroclada leaf extract, the okinawa mozuku extract and the beta glucan are formulated to support improved stem cell mobilization.

9. The composition of claim 5, wherein the synergistically effective amounts of the blue-green algae extract, the okinawa mozuku extract, the beta glucan, and the nucleotides are formulated to support improved stem cell migration.

10. The composition of claim 5, wherein the synergistically effective amounts of the L-serine, the chlorophyllin compound, the astragalus root extract, the Panax notoginseng root extract, the white peony root extract, the beta glucan, the salvia miltiorrhiza root extract, the nucleotides, the royal jelly, and the bromelain are formulated to support improved stem cell proliferation.

11. The composition of claim 5, wherein the synergistically effective amounts of the L-serine, the chlorophyllin compound, the ferulic acid, the astragalus root extract, the panax notoginseng root extract, the white peony root extract, the beta glucan, the royal jelly, the bromelain, and the salvia miltiorrhiza root extract are formulated to support improved stem cell differentiation.

12. The composition of claim 5, wherein the synergistically effective amounts of the L-serine, the ferulic acid, the Panax notoginseng root extract, the nucleotides, the white peony root extract, and salvia miltiorrhiza root extract are formulated to support improved stem cell self-renewal.

13. The composition of claim 5, wherein the synergistically effective amounts of the L-serine, the astragalus root extract, and the okinawa mozuku extract are formulated to support improved stem cell survival.

14. The composition of claim 1, further disposed within at least one pharmaceutically acceptable vehicle.

15. The composition of claim 1, wherein the composition is formulated to be taken orally:
one of the doses per day, for four consecutive days every month; or
one half of the dose twice a day, for four consecutive days every month.

16. The composition of claim 2, the dose of the composition further comprising a synergistically effective amount of one or more of:
Echinacea purpurea extract;
Angelica sinensis root extract;
Chlorella growth factor; and
Vitamin C.

17. The composition of claim 16, wherein the synergistically effective amount comprises:
approximately 100-500 mg of the Echinacea purpurea extract (400 mg);
approximately 100-500 mg of the Angelica sinensis root extract;
approximately 200-750 mg of the Chlorella growth factor (200 mg); and
approximately 50-500 mg of the Vitamin C (250 mg).

18. A method of supporting stem cell health in humans, the method comprising:
orally administering a composition for supporting stem cell health to an individual in need thereof, a dose of the composition comprising a synergistically effective amount of:
at least one of:
sea buckthorn fruit extract,
blue-green algae extract, and
Aloe macroclada leaf extract;
soluble beta glucan;
Panax notoginseng root extract;
royal jelly powder;
L-serine;
okinawa mozuku extract; and
nucleotides;
wherein the orally administering the composition comprises:
administering one of the doses per day, for four consecutive days every month; or
administering one half of the dose twice per day, for four consecutive days every month.

19. The method of claim 18, further comprising at least one of:
identifying the individual in need of stem cell health support;
evaluating or monitoring the stem cell function of the individual after the composition is orally administered; and
observing improved stem cell function compared to before the composition was orally administered to the individual.

20. A method of manufacturing a composition for supporting stem cell health in humans, the method comprising:
combining a synergistically effective amount of:
at least one of:
sea buckthorn fruit extract,
blue-green algae extract, and
Aloe macroclada leaf extract;
soluble beta glucan;
Panax notoginseng root extract;
royal jelly powder;
L-serine;
okinawa mozuku extract; and
nucleotides; and
disposing the composition within at least one pharmaceutically acceptable vehicle.
